SEQUENCE LISTING (63 sequences)

SEQ ID NO: 1 - DNA, 2100 bp, Homo sapiens
atgccctcgctgtggagagaaatcctcttggagtcgctgctgggatgtgtttcttggtctctctaccatgacctgggaccgatgatctattactttcctttgcaaacactagaactcactgggcttgaaggttttagtatagcatttctttctccaatattcctaacaattactcctttctggaaattggttaacaagaagtggatgctaaccctgctgaggataatcactattggcagcatagcctccttccaggctccaaatgccaaacttcgactgatggttcttgcgcttggggtgtcttcctcactgatagtgcaagctgtgacttggtggtcaggaagtcatttgcaaaggtacctcagaatttggggattcattttaggacagattgttcttgttgttctacgcatatggtatacttcactaaacccaatctggagttatcagatgtccaacaaagtgatactgacattaagtgccatagccacacttgatcgtattggcacagatggtgactgcagtaaacctgaagaaaagaagactggtgaggtagccacggggatggcctctagacccaactggctgctggcaggggctgcttttggtagccttgtgttcctcacccactgggtttttggagaagtctctcttgtttccagatgggcagtgagtgggcatccacatccagggccagatcctaacccatttggaggtgcagtactgctgtgcttggcaagtggattgatgcttccatcttgtttgtggtttcgtggtactggtttgatctggtgggttacaggaacagcttcagctgcggggctcctttacctgcacacatgggcagctgctgtgtctggctgtgtcttcgccatctttactgcatccatgtggccccaaacacttggacaccttattaactcagggacaaaccctgggaaaaccatgaccattgccatgatattttatcttctagaaatatttttctgtgcctggtgcacagcttttaagtttgtcccaggaggtgtctacgctagagaaagatcagatgtgcttttggggacaatgatgttaattatcgggctgaatatgctatttggtcctaagaaaaaccttgacttgcttcttcaaacaaaaaacagttctaaagtgcttttcagaaagagtgaaaaatacatgaaactttttctgtggctgcttgttggtgtgggattgttgggattaggactacggcataaagcctatgagagaaaactgggcaaagtggcaccaaccaaagaggtctctgctgccatctggcctttcaggtttggatatgacaatgaagggtggtctagtctagaaagatcagctcacctgctcaatgaaacaggtgcagatttcataacaattttggagagtgatgcttctaagccctatatggggaacaatgacttaaccatgtggctaggggaaaagttgggtttctatacagactttggtccaagcacaaggtatcacacttgggggattatggctttgtcaagatacccaattgtgaaatctgagcatcaccttcttccgtcaccagagggcgagatcgcaccagccatcacattgaccgttaacatttcgggcaagctggtggattttgtcgtgacacactttgggaaccacgaagatgacctcgacaggaaactgcaggctattgctgtttcaaaactactgaaaagtagctctaatcaagtgatatttctgggatatatcacttcagcacctggctccagagattatctacagctcactgaacatggcaatgtgaaggatatcgacagcactgatcatgacagatggtgtgaatacattatgtatcgagggctgatcaggttgggttatgcaagaatctcccatgctgaactgagtgattcagaaattcagatggcaaaatttaggatccctgatgaccccactaattatagagacaaccagaaagtggtcatagaccacagagaagtttctgagaaaattcattttaatcccagatttggatcctacaaagaaggacacaattatgaaaacaaccatcattttcatatgaatactcccaaatactttttatga

SEQ ID NO: 2 - protein, 699 aa, Homo sapiens
MPSLWREILLESLLGCVSWSLYHDLGPMIYYFPLQTLELTGLEGFSIAFLSPIFLTITPFWKLVNKKWMLTLLRIITIGSIASFQAPNAKLRLMVLALGVSSSLIVQAVTWWSGSHLQRYLRIWGFILGQIVLVVLRIWYTSLNPIWSYQMSNKVILTLSAIATLDRIGTDGDCSKPEEKKTGEVATGMASRPNWLLAGAAFGSLVFLTHWVFGEVSLVSRWAVSGHPHPGPDPNPFGGAVLLCLASGLMLPSCLWFRGTGLIWWVTGTASAAGLLYLHTWAAAVSGCVFAIFTASMWPQTLGHLINSGTNPGKTMTIAMIFYLLEIFFCAWCTAFKFVPGGVYARERSDVLLGTMMLIIGLNMLFGPKKNLDLLLQTKNSSKVLFRKSEKYMKLFLWLLVGVGLLGLGLRHKAYERKLGKVAPTKEVSAAIWPFRFGYDNEGWSSLERSAHLLNETGADFITILESDASKPYMGNNDLTMWLGEKLGFYTDFGPSTRYHTWGIMALSRYPIVKSEHHLLPSPEGEIAPAITLTVNISGKLVDFVVTHFGNHEDDLDRKLQAIAVSKLLKSSSNQVIFLGYITSAPGSRDYLQLTEHGNVKDIDSTDHDRWCEYIMYRGLIRLGYARISHAELSDSEIQMAKFRIPDDPTNYRDNQKVVIDHREVSEKIHFNPRFGSYKEGHNYENNHHFHMNTPKYFL

SEQ ID NO: 3 - protein, 2 aa, Homo sapiens
YF

SEQ ID NO: 4 - protein, 4 aa, Homo sapiens
KKKS

SEQ ID NO: 5 - protein, 22 aa, Artificial Sequence
  Synthetic
NTFYETFKTRSYWLGKSKKKSM

SEQ ID NO: 6 - DNA, 10 bp, Artificial Sequence
  Synthetic
accagccata

SEQ ID NO: 7 - protein, 4 aa, Artificial Sequence
  Synthetic
  misc_feature (3)..(4): Xaa can be any naturally occurring amino acid
KKXX

SEQ ID NO: 8 - DNA, 85 bp, Homo sapiens
cacaattatgaaaacaaccatcattttcatatgaatactcccatactttttatgaaacatttaaaacaagaagttattggctggg

SEQ ID NO: 9 - DNA, 85 bp, Homo sapiens
cacaattatgaaaacaaccatcattttcatatgaatactcccaatactttttatgaaacatttaaaacaagaagttattggctgg

SEQ ID NO: 10 - DNA, 85 bp, Homo sapiens
cacaattatgaaaaaccatcattttcatatgaatactcccaaatactttttatgaaacatttaaaacaagaagttattggctggg

SEQ ID NO: 11 - DNA, 84 bp, Homo sapiens
cacaattatgaaaacaaatcattttcatatgaatactcccaatactttttatgaaacatttaaaacaagaagttattggctggg

SEQ ID NO: 12 - DNA, 84 bp, Homo sapiens
cacaattatgaaaacaaccatcattttcatgaatactcccaatactttttatgaaacatttaaaacaagaagttattggctggg

SEQ ID NO: 13 - DNA, 84 bp, Homo sapiens
cacaattatgaaaacaaccatcattttcatatgaatactcccaatactttatgaaacatttaaaacaagaagttattggctggg

SEQ ID NO: 14 - DNA, 84 bp, Homo sapiens; identical to SEQ ID NO: 11

SEQ ID NO: 15 - DNA, 83 bp, Homo sapiens
cacaattatgaaaacaaccatcttcatatgaatactcccaatactttttatgaaacatttaaaacaagaagttattggctggg

SEQ ID NO: 16 - DNA, 85 bp, Homo sapiens
cacaattatgaaaacaaccatcattttcatatgaatactcccaaatactttatgaaacatttaaaacaagaagttattggctggg

SEQ ID NO: 17 - DNA, 84 bp, Homo sapiens
cacaattatgaaaacaaccatcattttcatatgaatactcccaatactttttatgaaacattaaacaagaagttattggctggg

SEQ ID NO: 18 - DNA, 85 bp, Homo sapiens
cacaattatgaaaacaaccatcattttcatatgaatactcccaaatactttttatgaaacatttaaaacaagaagttattgtggg

SEQ ID NO: 19 - DNA, 82 bp, Homo sapiens
cacaattatgaaaacaacattttcatatgaatactcccaatactttttatgaaacatttaaaacaagaagttattggctggg

SEQ ID NO: 20 - DNA, 84 bp, Homo sapiens
cacaattatgaaaacaaccatcattttcatatgaactcccaatactttttatgaaacatttaaaacaagaagttattggctggg

SEQ ID NO: 21 - DNA, 84 bp, Homo sapiens
cacaattatgaaaacaaccataattttcatatgaataccaaatactttttatgaaacatttaaaacaagaagttattggctggg

SEQ ID NO: 22 - DNA, 84 bp, Homo sapiens
cacaattatgaaaacaaccatcattttcatatgaatactcccaatactttttatgaaacatttaacaagaagttattggctggg

SEQ ID NO: 23 - DNA, 84 bp, Homo sapiens
cacaattatgaaaacaaccatcattttcatatgaatactcccaatactttttatgaaacatttaaaaagaagttattggctggg

SEQ ID NO: 24 - DNA, 84 bp, Homo sapiens
cacaattatgaaaacaaccatcattttcatattactcccaaatactttttatgaaacatttaaaacaagaagttattggctggg

SEQ ID NO: 25 - DNA, 83 bp, Homo sapiens
cacaattatgaaaacaaccataattttcatatgaatactcccaaatactttttatgaaataaaacaagaagttattggctggg

SEQ ID NO: 26 - DNA, 83 bp, Homo sapiens
cattatgaaaacaaccatcattttcatatgaatactcccaatactttttatgaaacatttaaaacaagaagttattggctggg

SEQ ID NO: 27 - DNA, 84 bp, Homo sapiens
cacaattatgaaaacaaccataattttcatatgaatactccctactttttatgaaacatttaaaacaagaagttattggctggg

SEQ ID NO: 28 - DNA, 83 bp, Homo sapiens
cacaattatgaaaacaaccatcattttcatatgaatactcccaatactttttatgaatttaaaacaagaagttattggctggg

SEQ ID NO: 29 - DNA, 87 bp, Homo sapiens
cacaattatgaaaacaaccatcattttcatatgaatactcccaaatactttttatgaaacatttaaaacaagaagttattggctggg

SEQ ID NO: 30 - protein, 18 aa, Homo sapiens
HNYENNHHFHMNTPKYFL

SEQ ID NO: 31 - DNA, 81 bp, Homo sapiens
tcaccttcttccgtcaccagagggcgagatcgcaccagccatcacttgaccgttaacacgggcaagctggtggattttgtc

SEQ ID NO: 32 - DNA, 82 bp, Homo sapiens
tcaccttcttccgtcaccagagggcgagatcgcaccagccatcacttgaccgttaacatttcggaagctggtggattttgtc

SEQ ID NO: 33 - DNA, 80 bp, Homo sapiens
tcaccttcttccgtcaccagagggctcgcaccagccatcacttgaccgttaacatttcgggcaagctggtggattttgtc

SEQ ID NO: 34 - DNA, 81 bp, Homo sapiens
tcaccttcttccgtcaccagagggcgagatcgcaccagccatctgaccgttaacatttcgggcaagctggtggattttgtc

SEQ ID NO: 35 - DNA, 81 bp, Homo sapiens
tcaccttcttccgtcaccagagggcgagatcgcaccagccatcacattgaccgtatttcgggcaagctggtggattttgtt

SEQ ID NO: 36 - DNA, 80 bp, Homo sapiens
tcaccttcttccgtcaccagagggcgagatcgcaccagccatcacttgaccgttaacatttcgggcaagctggtggattt

SEQ ID NO: 37 - DNA, 82 bp, Homo sapiens
tcaccttcttccgtcaccagagggcgagatcgcaccagccatcacattgaccgttatttcgggcaagctggtggattttgtc

SEQ ID NO: 38 - DNA, 82 bp, Homo sapiens
tccttcttccgtcaccagagggcgagatcgcaccagccatcacttgaccgttaacatttcgggcaagctggtggattttgtc

SEQ ID NO: 39 - DNA, 82 bp, Homo sapiens
tcactcttccgtcaccagagggcgagatcgcaccagccatcacttgaccgttaacatttcgggcaagctggtggattttgtc

SEQ ID NO: 40 - DNA, 83 bp, Homo sapiens
tcactcttccgtcaccagagggcgagatcgcaccagccatcacattgaccgttaacatttcgggcaagctggtggattttgtc

SEQ ID NO: 41 - DNA, 82 bp, Homo sapiens
tcaccttctcgtcaccagagggcgagatcgcaccagccatcacttgaccgttaacatttcgggcaagctggtggattttgtc

SEQ ID NO: 42 - DNA, 76 bp, Homo sapiens
tcaccttcttccgtcaccagagggcgagatcgcaccagccatcacttgaccgttaacatttcgggcaagctggttc

SEQ ID NO: 43 - DNA, 76 bp, Homo sapiens
tcaccttcttccgtcaccagagggcgagatcgcaccagccatcacttgaccgttaacatttcgggcaagctggtgg

SEQ ID NO: 44 - DNA, 80 bp, Homo sapiens; identical to SEQ ID NO: 36

SEQ ID NO: 45 - DNA, 83 bp, Homo sapiens
tccttcttccgtccccagagggcgagatcgcaccagccatcacattgaccgttaacatttcgggcaagctggtggattttgtc

SEQ ID NO: 46 - DNA, 80 bp, Homo sapiens
tcaccttcttccccagagggcgagatcgcaccagccatcacttgaccgttaacatttcgggcaagctggtggattttgtc

SEQ ID NO: 47 - DNA, 78 bp, Homo sapiens
tcaccttcttccgtcaccagagggcgagatcgcatcacattgaccgttaacatttcgggcaagctggtggattttgtc

SEQ ID NO: 48 - DNA, 85 bp, Homo sapiens
tcaccttcttccgtcaccagagggcgagatcgcaccagccatcacattgaccgttaacatttcgggcaagctggtggattttgtc

SEQ ID NO: 49 - DNA, 81 bp, Homo sapiens
tcaccttattccgtcaccaggcgagatcgcaccagccatcacttgaccgttaacatttcgggcaagctggtggattttgtc

SEQ ID NO: 50 - DNA, 78 bp, Homo sapiens
tcaccttcttccgtcaccagagggcgagatcgcaccagccatcacttgaccgttaacatttcgggcaagctggtggtc

SEQ ID NO: 51 - DNA, 82 bp, Homo sapiens
tcaccttcttccgtcaccagagggcgatcgcaccagccatcacttgaccgttaacatttcgggcaagctggtggattttgtc

SEQ ID NO: 52 - DNA, 80 bp, Homo sapiens
tcaccttcttccgtcaccagagggcgagatcgcaccagccatcactcgttaacatttcgggcaagctggtggattttgtc

SEQ ID NO: 53 - DNA, 85 bp, Homo sapiens; identical to SEQ ID NO: 48

SEQ ID NO: 54 - protein, 28 aa, Homo sapiens
HLLPSPEGEIAPAITLTVNISGKLVDFV

SEQ ID NO: 55 - DNA, 146 bp, Homo sapiens
atggtgctgtctcggtacccgattgtcagatcggaacatcaccttcttccgtcgccagagggcgagatcgcaccagccataaccatgacagttaacgtctccaacagactggtggattttgtggtgacacactttgggaatcatga

SEQ ID NO: 56 - protein, 48 aa, Homo sapiens
MVLSRYPIVRSEHHLLPSPEGEIAPAITMTVNVSNRLVDFVVTHFGNH

SEQ ID NO: 57 - DNA, 145 bp, Homo sapiens
atggtgctgtctcggtacccgattgtcagatcggaacatcaccttcttccgtcgccagagggcgagatcgcaccagccataacgtgacagttaacgtctccaacagactggtggattttgtggtgacacactttgggaatcatga

SEQ ID NO: 58 - protein, 46 aa, Homo sapiens
MVLSRYPIVRSEHHLLPSPEGEIAPAITQLTSPTDWWILWHTLGIM

SEQ ID NO: 59 - DNA, 10 bp, Homo sapiens
accagccata

SEQ ID NO: 60 - DNA, 145 bp, Homo sapiens
atggtgctgtctcggtacccgattgtcagatcggaacatcaccttcttccgtcgccagagggcgagatcgcaccagccataaccatgacagttaacgtctccaacagactggtggattttgtggtgacacactttgggaatcatg

SEQ ID NO: 61 - protein, 43 aa, Homo sapiens
MVLSRYPIVRSEHHLLPSPEGEIAPQLTSPTDWWILWHTLGIM

SEQ ID NO: 62 - protein, 10 aa, Homo sapiens
HHMNTPKYFL

SEQ ID NO: 63 - protein, 28 aa, Homo sapiens
HHMNTPNTFYETFKTRSYWLGKSKKKSM

(19) United States
(12) Patent Application Publication
(10) Pub. No.: US 2022/0283183 A1
(43) Pub. Date: Sep. 8, 2022

(54) DIAGNOSTIC TEST FOR IDIOPATHIC NORMAL PRESSURE HYDROCEPHALUS

(71) Applicant: University of Massachusetts, Boston, MA (US)

(72) Inventor: Mark D. Johnson, Sudbury, MA (US)

(73) Assignee: University of Massachusetts, Boston, MA (US)

(21) Appl. No.: 17/634,586

(22) PCT Filed: Aug. 12, 2020
(86) PCT No.: PCT/US20/45879

§ 371 Date: Feb. 11, 2022

Related U.S. Application Data
(60) Provisional application No. 62/885,792, filed on Aug. 12, 2019.

(51) Int. Cl. G01N 33/68; A01K 67/027; C12Q 1/6883

(52) U.S. Cl. CPC G01N 33/6896; A01K 67/0276; C12Q 1/6883; A01K 2217/075; C12Q 2600/156; G01N 2800/2814

(57) ABSTRACT

Aspects of the disclosure relate to methods and compositions for diagnosing and/or treating idiopathic Normal Pressure Hydrocephalus (iNPH). In some embodiments, the methods comprise detecting a level of Cwh43 gene expression or Cwh43 protein in a subject and administering to the subject one or more therapies to treat iNPH based upon the level of the Cwh43 gene expression or Cwh43 protein compared to a control sample.

### CROSS-REFERENCE TO RELATED APPLICATIONS

This application is a national stage filing under 35 U.S.C. § 371 of international PCT application PCT/US2020/045879, filed Aug. 12, 2020, which claims priority under 35 U.S.C. § 119(e) to U.S. provisional patent application, U.S. Ser. No. 62/885,792, filed Aug. 12, 2019, the entire contents of each of which are incorporated herein by reference.

### STATEMENT REGARDING FEDERALLY SPONSORED RESEARCH OR DEVELOPMENT

This invention was made with government support under grant numbers NS100511 and NS106985 awarded by the National Institutes of Health. The government has certain rights in the invention.

### BACKGROUND

Idiopathic normal pressure hydrocephalus (iNPH) is a neurological disorder of aging that is characterized by enlarged cerebral ventricles, gait difficulty, incontinence, and dementia. Because the symptoms can be improved by cerebrospinal fluid (CSF) drainage, iNPH is classified as a reversible dementia. Idiopathic normal pressure hydrocephalus affects about 700,000 Americans and occurs almost exclusively after the age of 60. Many patients are misdiagnosed or undiagnosed, in part because the symptoms of iNPH resemble the symptoms of other neurological disorders such as Parkinson's Disease or Alzheimer's Disease.

### SUMMARY

Aspects of the disclosure relate to methods and interventions useful in the diagnosis and treatment of iNPH. In some aspects, the disclosure relates to compositions and methods for the diagnosis and treatment of iNPH.

In some embodiments, the methods involve detecting in a biological sample obtained from a subject (e.g., a mammalian subject, such as a human subject), the presence of certain nucleotide mutations, substitutions, insertions, or deletions (e.g., mutations, substitutions, insertions, or deletions of a CWH43 gene of a subject). In some embodiments the one or more mutations, substitutions, insertions, or deletions results in a heterozygous CWH43 deletion (e.g., a nucleotide deletion) that disrupts the carboxyl terminus of the Cwh43 protein (e.g., causes a frameshift mutation inserting a stop codon resulting in truncated Cwh43 protein). In some embodiments, the one or more mutations are set forth in Table 1. In some embodiments, the one or more mutations, substitutions, insertions, or deletions results in translation of a truncated gene product (e.g., a truncated Cwh43 protein) or reduced expression of a gene product (e.g., a deletion of a CWH43 gene resulting in reduction of Cwh43 protein translation in a subject). In some embodiments, methods involve detecting one or more proteins, such as one or more gene products of a CWH43 gene (e.g., full-length Cwh43 protein, truncated Cwh43 protein, etc.). In some embodiments, methods further comprise administering to the subject a therapeutic agent (e.g., a nucleic acid, peptide, small molecule, or any combination thereof) or performing a surgical procedure on a subject (e.g., CSF drainage).

In some aspects, the disclosure relates to a method of diagnosing idiopathic normal pressure hydrocephalus (iNPH) in a subject, comprising: (a) measuring in a biological sample from the subject a level of Cwh43 protein; and (b) comparing the level of Cwh43 protein in the sample from the subject with a control level of Cwh43 protein. In some aspects, the method of diagnosing further comprises a treatment comprising: (c) administering to the subject at least one treatment for iNPH when the level of Cwh32 protein is lower than the control level of Cwh43 protein.

In some aspects, the disclosure relates to a method of diagnosing idiopathic normal pressure hydrocephalus (iNPH) in a subject, comprising: (a) analyzing the CWH43 gene in a sample from a subject; and (b) identifying one or more mutations that negatively affect ER export signal of CWH43. In some aspects, the method of diagnosing further comprises a treatment comprising: (c) administering at least one treatment for iNPH if the CWH43 gene product has at least one mutation negatively affecting the ER export signal of the CWH43 gene product.

In some aspects, the disclosure relates to a method of diagnosing idiopathic normal pressure hydrocephalus (iNPH) in a subject, comprising: (a) identifying a truncated Cwh43 protein in a biological sample obtained from a subject. In some aspects, the method of diagnosing further comprises a treatment comprising: (b) administering at least one treatment for iNPH if the isolated Cwh43 is truncated compared to the control Cwh43 protein.

In some embodiments, the analysis performed in any of the methods of the disclosure comprise sequencing the exome of the subject.

In some embodiments, a mutation according to any of the methods of the disclosure results in a truncation of the CWH43 gene product within 1, 2, 3, 4, 5, 6, 7, 8, 9, 10 residues, or at residue 533. In some embodiments, in any of the methods of the disclosure an isolated Cwh43 protein from a sample from a subject is truncated within 1, 2, 3, 4, 5, 6, 7, 8, 9, 10 residues, or at residue 533. In some embodiments, in any of the methods of the disclosure a mutation in the CWH43 gene which negatively affect the ER export signal is the result of a termination codon being introduced into the gene such that the Cwh43 protein terminates within 1, 2, 3, 4, 5, 6, 7, 8, 9, 10 residues, or at residue 533. In some embodiments, in any of the methods of the disclosure a mutation is one of the mutations listed in Table 1. In some embodiments, a CWH43 gene according to any of the methods of the disclosure, comprises a Lys696AsnfsTer23 mutation.

In some embodiments, the measuring as performed in any of the methods described herein, comprises an antibody-based assay. In some embodiments, the antibody used in the antibody-based assay, comprises a detection tag or moiety.

In some embodiments, any of the methods described herein are performed in connection with at least one additional method for determining the risk of iNPH in the subject. In some embodiments, the at least one additional method comprises a method selected from: evaluation for symmetric gait disturbances, evaluation for dementia, evaluation for incontinence, and a negative determination of other causes of hydrocephalus.

In some embodiments, the at least one treatment of any of the methods disclosed herein, comprises, cerebral spinal fluid (CSF) drainage. In some embodiments, the CSF drainage is performed via implantation of a shunt. In some embodiments, the at least one treatment of any of the methods disclosed herein, comprises, administration of an exogenous Cwh43 protein with at least 70% identity to wild-type Cwh43 protein (SEQ ID NO: 2). In some embodiments, the wild-type Cwh43 protein is human Cwh43 protein.

In some embodiments, the subject of any of the methods disclosed herein, is a mammal. In some embodiments, the mammal is human. In some embodiments, the subject of any of the methods of the disclosure is at least 45 years of age. In some embodiments, the subject is between about 40 and 55 years of age. In some embodiments, the subject of any of the methods of the disclosure exhibits at least one other symptom of iNPH. In some embodiments, the subject exhibits at least two other symptoms of iNPH. In some embodiments, the subject exhibits at least three other symptoms of iNPH.

### BRIEF DESCRIPTION OF DRAWINGS

Further aspects of the disclosure will be readily appreciated upon review of the Detailed Description of its various aspects and embodiments, described below, when taken in conjunction with the accompanying Drawings.

FIGS. 1A-1D show that idiopathic Normal Pressure Hydrocephalus (iNPH)-associated deletions disrupt the ability of Cwh43 to regulate the localization of glycosylphosphatidylinositol (GPI)-anchored receptors. FIG. 1A shows Sanger DNA sequencing data from two iNPH patients confirming the presence of heterozygous CWH43 deletions. Arrows identify location of the deletion. The nucleotide identifier above a peak corresponds to the top-lined (e.g., upper-most line at that position) peak. FIG. 1B shows a diagram of the domain structure of the Cwh43 protein illustrating the location of the damaging CWH43 deletions. Lower panel details the disruptive effect of the K696Asnfs mutation on the C-terminal endoplasmic reticulum (ER) export signal (SEQ ID NO: 3), replacing it via a frameshift with a 23 amino acid sequence that contains an ER retention signal (SEQ ID NO: 4). Sequences shown in FIG. 1B correspond to SEQ ID NO: 62 and 63, from top to bottom. FIG. 1C shows fluorescence micrographs of HeLa cells transfected with plasmids encoding human green fluorescent protein (GFP)-Cwh43 or GFP-Cwh43-K696fs fusion proteins. Arrows identify intracellular vesicles. Note the effect of the K696fs mutation on the association of Cwh43 with the Golgi apparatus and intracellular vesicles. Scale is approximately 5 micrometers (μm). FIG. 1D shows Western blot analysis of total membrane, aqueous and lipid (GPI-anchor-containing) Triton X-114 extracts derived from wild-type HeLa cells and two independent CRISPR CWH43 knockout (KO) HeLa cell lines in which a mutated CWH43 gene encodes a protein that is truncated near Leu533 and CWH43 messenger RNA (mRNA) and protein are markedly reduced. Cells were transfected to overexpress a control GFP plasmid, a plasmid encoding human wild-type Cwh43 with GFP fused to the N-terminus, or a plasmid encoding human CWH43 harboring the iNPH-associated mutation (Lys696AsnfsTer23) with GFP fused to the N-terminus. The Western blot was stained using an antibody directed against CD59, a GPI-anchored protein.

FIGS. 2A-2C shows expression of Cwh43 mRNA and protein in the mouse brain. FIG. 2A illustrates mRNA in situ hybridization images showing expression of Cwh43 mRNA in the mouse brain. Enclosed areas containing portions of the ventricle, hippocampus, and dorsal thalamus are shown at higher magnification on the right. Arrowheads point to choroid plexus. FIG. 2B shows fluorescence immunohistochemistry of the ependymal surface of the lateral ventricle of the mouse brain. Cilia are visualized using an antibody for acetylated alpha tubulin (panel 1 from left to right). Cwh43 is visualized using a specific anti-Cwh43 antibody (panel 2 from left to right). Nuclei are counterstained using DAPI (panel 3 from left to right). A composite of all three previous panels (panel 4 from left to right). Scale is approximately 25 μm. FIG. 2C shows confocal fluorescence immunocytochemistry images of a single cultured mouse ciliated ependymal cell. Cilia were visualized using an antibody for acetylated alpha tubulin (panel 2 from top to bottom). Cwh43 immunoreactivity was visualized using a specific anti-Cwh43 antibody (panel 1 from top to bottom). A composite of all panels 1 and 2 (panel 3 from top to bottom). Scale (left column) is approximately 4 μm. Images in the column on the right represent a Z-stack reconstruction of confocal images showing localization of Cwh43 immunoreactivity in cilia of a mouse ependymal cell. Scale (right column) is approximately 5 μm.

FIGS. 3A-3D show that CWH43 mutations can increase ventricular volume and causes gait dysfunction in mice. FIG. 3A is a scatter plot comparing ventricular volume from CWH43^{WT/WT} (wild-type, WT), heterozygous CWH43^{WT/M533}, homozygous CWH43^{M533/M533} and CWH43^{M533/A530} mice at 6 months. Ventricular volume was calculated from T2-weighted MR images of the mouse brain using a custom automated computer algorithm. Horizontal bars indicate the mean of the measurements in each column. Statistical significance for each mutant mouse line compared to WT was determined using the unpaired t-test (P<0.0015 for CWH43^{WT/M533}; P<0.0014 for CWH43^{M533/M533}; P<0.0065 for CWH43^{M533/A530}). FIG. 3B shows representative 3D volumetric MR images of mouse brains from 6-month-old CWH43^{WT/WT}, CWH43^{WT/M533}, CWH43^{M533/M533}, and CWH43^{M533/A530} mice. LV=lateral ventricle, 3rd V=third ventricle, 4th V=fourth ventricle. FIG. 3C shows quantitative gait analysis at 7-months of age revealed increased sway among homozygous CWH43^{M533/M533} mice (P<0.04, n=7, unpaired t-test) and compound heterozygous CWH43^{M533/A530} (P<0.05, n=4, unpaired t-test) mice when compared to wild-type CWH43 ^{WT/WT} mice (n=10). Sway (the distance between the hind paws during walking) was measured repeatedly for individual mice in each group during a constrained unidirectional walk. Data shown are the mean±SD for each group. FIG. 3D is a box plot showing rotarod performance data for CWH43^{WT/WT,} CWH43^{WT/M533}, CWH43^{M533/M533}, and CWH43^{M533/A530} mice at 7-months of age. Data shown are the mean, 1st quartile, 3rd quartile, minimum, and maximum for each group of mice.

When compared to wild-type CWH43^{WT/WT} mice (n=10), balance time on the rotarod was decreased significantly among heterozygous CWH43^{WT/M533} mice (P<0.0405, n=8, unpaired t-test), homozygous CWH43^{M533/M533} mice (P<0.0323, n=7, unpaired t-test), and compound heterozygous CWH43^{M533/A530} mice (P<0.0301, n=4, unpaired t-test).

FIGS. 4A-4C show that the CWH43 mutation decreases cilia number and alters the distribution of GPI-anchored proteins in mouse ventricular ciliated epithelia. FIG. 4A shows scanning electron micrographs of the ependymal surface of the lateral ventricle of CWH43 WT and CWH43^{M533/M533} mice. Scale is approximately 40 μm. The graph on the right quantifies the data from the electron micrographs on the left. Data shown are the mean±SEM. *=P<0.0037, n=4, unpaired t-test. FIG. 4B shows a Western blot analysis of total membrane, aqueous and lipid (GPI-anchor- containing) Triton X-114 extracts derived from wild-type or CWH43^{M533/M533} mouse brain or kidney. The Western blot was stained using an antibody directed against CD59, a GPI-anchored protein. FIG. 4C shows fluorescence immunohistochemistry for CD59 in the choroid plexus of the lateral ventricle from CWH43^{WT/WT} and CWH43^{M533/M533} mice. Arrowheads point to apical surfaces of choroid plexus cells. Nuclei are counterstained using DAPI (blue). Scale is approximately 5 μm.

FIG. 5 is a screen shot of sequencing data for CWH43:NM_001286791:exon16:c.2005delA:p.K696fs. Interactive Genome Viewer visualization showing reads used to call this variant. Data shown are re-assembled reads produced by GATK HaplotypeCaller-bamOutput. The data accurately represent what HaplotypeCaller was seeing when it called this variant. Sequences correspond to SEQ ID NO: 8-30, from top to bottom.

FIG. 6 is a screen shot of sequencing data for CWH43:NM_001286791:exon12:c.1515delA:p.Leu533Ter. Interactive Genome Viewer visualization showing reads used to call this variant. Data shown are re-assembled reads produced by GATK HaplotypeCaller-bamOutput. The data accurately represent what HaplotypeCaller was seeing when it called this variant. Sequences correspond to SEQ ID NO: 31-53, from top to bottom.

FIG. 7 shows that the CWH43 Leu533Ter deletion alters the subcellular distribution of Cwh43 protein in human cells. Fluorescence micrographs of human HeLa cells transfected with plasmids encoding GFP-Cwh43 or GFP-Cwh43-Leu533Ter. Note the effect of the Leu533Ter deletion on the association of Cwh43 with the endoplasmic reticulum, Golgi apparatus and intracellular vesicles. When compared to the full length GFP-Cwh43 protein. Scale is approximately 5 μm.

FIGS. 8A-8C show that the iNPH-associated CWH43 mutation decreases vesicular association but not cell surface expression of CD59. FIG. 8A depicts a Western blot showing loss of Cwh43 protein expression in two independent HeLa cell lines (KO1 and KO2) in which the CWH43 gene has been disrupted at Leu533 using CRISPR/Cas9 technology. FIG. 8B depicts fluorescence micrographs showing subcellular localization of two GPI-anchored proteins in wild-type (parental) or CWH43 knockout (CWH43 KO) HeLa cells. Cells were transiently transfected with plasmids encoding human RFP-CD59 or RFP-folate receptor alpha fusion proteins. Scale is approximately 5 μm. FIG. 8C depicts flow cytometry data showing cell surface CD59 expression in control (wild-type) and CWH43 knockout HeLa cell lines. Cells were labeled using an anti-CD59 antibody conjugated to a fluorophore.

FIG. 9 shows that Cwh43 protein is highly expressed in mouse choroid plexus. Immunofluorescence immunohistochemistry of the choroid plexus of the lateral ventricle of the mouse brain. Left panel shows cilia are visualized using an antibody for acetylated alpha tubulin. Center panel shows Cwh43 is visualized using a specific anti-Cwh43 antibody. Right panel shows a composite view of the left and center panels. Scale is approximately 25 μm.

FIG. 10 shows CRISPR/Cas9 generation of mutant CWH43 alleles in mice. CRISPR/Cas9 and CWH43 guide RNA injection into mouse embryos was used to generate two independent lines of C57b16 CWH43 mutant mice. Analysis of mouse DNA using RT-PCR and Sanger sequencing confirmed a 1 base pair deletion in mouse CWH43 (Met533Ter) corresponding to human 4:49034669 CA/C; Leu533Ter. A second mouse line) (CWH43^{M533/A530}) harboring one CWH43^{M533} allele and a second CWH43 allele with a 10 base pair deletion that generates a stop codon and termination of Cwh43 at A530. Sequences correspond to SEQ ID NO: 54-61, from top to bottom.

FIGS. 11A-11D show that the CWH43 mutation decreases cilia number in CWH43^{M533/A530} mice. FIG. 11A shows scanning electron micrographs of the ependymal surface of the lateral ventricle of CWH43 WT and CWH43^{M533/A530} mice. Scale is approximately 40 μm. FIG. 11B is a graph which quantifies the data from the electron micrographs on the left. Data shown are the mean±SEM. *=P<0.0037, n=4, unpaired t-test. FIG. 11C shows immunofluorescence immunohistochemistry of the ependymal surface of the lateral ventricle of the mouse brain. Cilia are visualized using an antibody for acetylated alpha tubulin (green). The tight junction protein, ZO-1 is visualized using a specific anti-ZO-1 antibody (red). Scale is approximately 5 μm. FIG. 11D is a graph showing quantitation of the number of cilia/unit area along the ventricular surface of wild-type (WT) and mutant CWH43^{M533/A530} mice. Data shown are the mean±SEM. *=P<0.0001, n=6, unpaired t-test.

FIG. 12 shows fluorescence immunohistochemistry for the GPI-anchored protein, CD59, in the ependyma of the lateral ventricle from CWH43^{WT/WT} and CWH43^{M533/M533} mice. Arrowheads point to apical surfaces of choroid plexus cells. Nuclei are counterstained using DAPI (blue). Scale is approximately 5 μm.

FIG. 13 is a table summarizing gene alterations identified.

### DETAILED DESCRIPTION

Aspects of the disclosure relate to compositions and methods for the diagnosis and treatment of idiopathic normal pressure hydrocephalus (iNPH). Idiopathic normal pressure hydrocephalus (also sometimes referred to as “primary” normal pressure hydrocephalus) is known as a syndrome characterized by gait impairment, cognitive decline, and urinary incontinence, and is associated with ventriculomegaly (e.g., hydrocephalus, a condition in which the cerebrospinal fluid (CSF) filled structures of the brain become enlarged) in the absence of elevated cerebrospinal fluid (CSF) pressure. As such, there is excess cerebrospinal fluid in the structures (e.g., ventricles) of the brain with normal to slightly elevated CSF pressure. It is believed that as the fluid builds up, it causes the ventricles to enlarge and the pressure inside the head to increase, compressing surrounding brain tissue, leading to neurological complications leading to the presentation of the “classic triad of symptoms” (e.g., urinary incontinence, dementia, and gait deviations).

Herein, it is shown that certain mutations related to the CWH43 gene, which encodes the Cwh43 protein, are found to be associated with iNPH. In yeast, Cwh43 incorporates ceramide into the lipid anchor of glycosylphosphatidylinositol (GPI) anchored proteins, thereby regulating their membrane localization. Herein, it is shown that Cwh43 regulates the membrane localization of GPI-anchored proteins in mammalian cells, and further that alterations to CWH43 can modulate this function. Specifically, mutations can disrupt this function.

Accordingly, In some embodiments, the methods involve detecting in a biological sample obtained from a subject (e.g., a mammalian subject, such as a human subject), the presence of certain nucleotide mutations (e.g., mutations, substitutions, insertions, or deletions of a CWH43 gene of a subject). A CWH43 gene, as may be used in any of the methods as described herein, may be any CWH43 gene of a subject. In some embodiments, a CWH43 gene may be a human CWH43 gene. In some embodiments, the human CWH43 gene may be any variant of a human CWH43 gene in a human. In some embodiments, the human CWH43 gene is a wild-type CWH43 gene. In some embodiments, the human CWH43 gene is represented by a nucleic acid sequence of GenBank Accession and Version number NC_000004.12 (SEQ ID NO: 1), which accession, and version number as well as the sequences associated therewith are incorporated herein by reference in their entirety. In some embodiments, the CWH43 gene may be a CWH43 gene with at least 70% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity, or more to SEQ ID NO: 1. In some embodiments, the CWH43 gene may be a CWH43 gene with a sequence comprising SEQ ID NO: 1.

The term “subject,” as used herein, refers to any organism in need of treatment or diagnosis using the subject matter herein. For example, without limitation, subjects may include mammals and non-mammals. In some embodiments, a subject is mammalian. In some embodiments, a subject is non-mammalian. As used herein, a “mammal,” refers to any animal constituting the class Mammalia (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, a mammal is a human. In some embodiments, the subject is an adult (e.g., mature) subject. In some embodiments the subject is at least 35 years of age. In some embodiments the subject is at least 40 years of age. In some embodiments the subject is at least 43 years of age. In some embodiments the subject is at least 45 years of age. In some embodiments the subject is at least 50 years of age. In some embodiments the subject is at least 55 years of age. In some embodiments the subject is at least 60 years of age. In some embodiments the subject is at least 65 years of age. In some embodiments the subject is at least 70 years of age. In some embodiments, the subject is less than 100 years of age. In some embodiments, the subject is less than 100 years of age. In some embodiments, the subject is less than 100 years of age. In some embodiments, the subject is less than 90 years of age. In some embodiments, the subject is less than 80 years of age. In some embodiments, the subject is less than 70 years of age. In some embodiments, the subject is less than 65 years of age. In some embodiments, the subject is less than 60 years of age. In some embodiments, the subject is less than 55 years of age. In some embodiments, the subject is less than 50 years of age. In some embodiments, the subject is less than 45 years of age. In some embodiments, the subject is less than 43 years of age. In some embodiments, the subject is less than 40 years of age.

In some embodiments, a subject may have, be at risk of having, or suspected of having iNPH. Accordingly, in some embodiments, a subject may exhibit one or more symptoms of iNPH. In some embodiments, a symptom is any symptom of indication associated with iNPH. There is significant variation in the clinical presentation and progression of this disorder, and its diagnosis often represents a challenge for medical professionals (e.g., neurologists, neurosurgeons). Gait deviations present in most patients and usually is the first symptom to appear. Typically, the gait abnormality is observed as a broad-based, slow, short-stepped, “stuck to the floor” movement and may resemble a gait associated with Parkinson's disease. The gait abnormalities are believed likely to be due to expansion of the lateral ventricles which impinge on the cortico spinal tract motor fibers. The gait deviation can be classified as mild, marked (e.g., the patient has difficulty walking because of considerable instability), or severe (e.g., it is not possible for the patient to walk without aid). In some embodiments, a symptom of iNPH comprises a gait deviation. In some embodiments, a gait deviation is mild. In some embodiments, a gait deviation is marked. In some embodiments, a gait deviation is severe.

Dementia presents as progressive cognitive impairment present in about 60% of patients at time of treatment. Impairment is observed in areas involving planning, organization, attention, and concentration, progressing to difficulty managing finances, taking medications, driving, keeping track of appointments, daytime sleeping, short-term memory impairments, and psychomotor slowing, and finally to later signs include apathy, reduced drive, slowed thinking, and reduced speech. It is believed this is due to distortions predominantly at the frontal lobe and the subcortex. In some embodiments, a symptom of iNPH comprises dementia. The dementia may be assessed by any means known in the art and will be readily apparent to the skilled artisan. In some embodiments, dementia may be indicated by observing impairment in any of the following areas: planning, organization, attention, concentration, difficulty managing finances, taking medications, driving, keeping track of appointments, daytime sleeping, short-term memory impairments, and psychomotor slowing in a subject. In some embodiments, dementia may be indicated by observing signs including apathy, reduced drive, slowed thinking, and reduced speech in a subject.

Urinary incontinence appears often later in iNPH and is present in approximately 50% of patients at time of treatment. It begins as increased frequency of urination and progresses to urge incontinence and permanent incontinence. In some embodiments, a symptom of iNPH comprises urinary incontinence. In some embodiments, the incontinence presents as an increased frequency of urination. In some embodiments, incontinence presents as an urge incontinence. In some embodiments, incontinence presents as permanent incontinence.

However, a simple diagnostic for iNPH does not presently exist. Evidenced-based diagnostic criteria for primary iNPH are: onset after age 40 years, duration of symptoms greater than 3 months; gait or balance impairment; and cognitive or urinary impairment. As such patients with suspected iNPH typically have the classical symptoms in addition to ventricular enlargement on neuroimaging. Imaging by magnetic resonance imaging (MRI) or computed tomography (CT) is often needed to demonstrate enlarged ventricles in the absence of macroscopic obstruction to cerebrospinal fluid flow. In some embodiments, a symptom of iNPH comprises ventricular enlargement. In some embodiments the ventricular enlargement is observed by neuroimaging. In some embodiments, the neuroimaging is by CT or MRI. In some embodiments, a ventricular enlargement is present in the absence of a macroscopic obstruction to the CSF. In some embodiments, a symptom of iNPH presents for a duration of greater than 1 month (e.g., 1, 2, 3, 4, 5, 6, or more months).

Treatments for iNPH typically comprise, CSF shunting (e.g., CSF drainage), which often provides significant symptom improvement in the majority of appropriately evaluated patients.). In addition, various supplemental tests, including a CSF “tap test,” external CSF drainage (often via spinal catheter), and CSF outflow resistance determination, can improve the accuracy of predicting a response to surgical treatment, however, there is at present not a single diagnostic test available for iNPH. The terms “treatment,” “treat,” and “treating,” as may be used interchangeably herein, refer to partially or completely alleviating, ameliorating, relieving, delaying onset of, inhibiting progression of, reducing severity of, and/or reducing incidence of one or more symptoms or features of a particular indication, disease, disorder, condition, and/or symptom thereof. In some embodiments, the treatment refers to a clinical intervention. In some embodiments, treatment comprises a surgical intervention (e.g., insertion, implantation of a shunt). As mentioned above, in some embodiments, treatments for iNPH may comprise, CSF shunting (e.g., CSF drainage), which often provides significant symptom improvement in the majority of appropriately evaluated patients.). In addition, various supplemental tests, including a CSF “tap test,” external CSF drainage (often via spinal catheter), and CSF outflow resistance determination, can improve the accuracy of predicting a response to surgical treatment, however, there is at present not a single diagnostic test available for iNPH. In some embodiments, a supplemental test is performed to assist in predicting the response to a treatment. In some embodiments, the supplemental test comprises: a CSF “tap test,” external CSF drainage, CSF outflow resistance determination, or combination thereof.

In some embodiments, treatment may be administered after one or more symptoms have developed and/or after a disease has been diagnosed. In other embodiments, treatment may be administered in the absence of symptoms (e.g., to prevent or delay onset of a symptom or inhibit onset or progression of a disease). For example, treatment may be administered to a susceptible individual (e.g., subject) prior to the onset of symptoms (e.g., in light of a history of symptoms and/or in light of genetic or other susceptibility factors). In some embodiments, the treatment is used and/or administered as a prophylaxis. Treatment may also be continued after symptoms have resolved, for example, to prevent or delay their recurrence. In some embodiments, the treatment is a treatment for iNPH. In some embodiments, the treatment comprises draining a portion of the subject's cerebrospinal fluid. In some embodiments, the treatment is the use of a cerebrospinal fluid shunt. In some embodiments, the treatment is the implantation of a cerebrospinal fluid shunt. In some embodiments, the at least one treatment in any of the methods of the disclosure comprises, administration of an exogenous Cwh43 protein with at least 70% identity to wild-type Cwh43 protein. In some embodiments, the wild-type Cwh43 protein is human Cwh43 protein.

In some embodiments, a CWH43 gene, as may be used in any of the methods as described herein, encodes a Cwh43 protein. In some embodiments, the Cwh43 protein is a human Cwh43 protein. In some embodiments, the Cwh43 protein is a wild-type human Cwh43 protein. In some embodiments, the Cwh43 protein comprises an amino acid sequence with at least 70% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99 or more identity) to SEQ ID NO: 2. In some embodiments, the Cwh43 protein comprises the amino acid sequence of SEQ ID NO: 2. In some embodiments, a CWH43 gene may encode a truncated Cwh43 protein. A truncation is the formation of a protein of fewer amino acid residues as compared to a reference protein. In some embodiments, a reference protein comprises a full-length Cwh43 protein from the same species of the subject. In some embodiments, a reference protein comprises a Cwh43 protein from a mammal. In some embodiments, a Cwh43 protein is from a human. In some embodiments, a Cwh43 protein is a wild-type Cwh43 protein. In some embodiments, a wild-type protein comprises a sequence with at least 70% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99 or more identity) to SEQ ID NO: 2. In some embodiments, a Cwh43 protein comprise the sequence of SEQ ID NO: 2. In some embodiments, a truncation results in a protein which is at least 1 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more) amino acid fewer in number than the reference protein. In some embodiments, a truncation results in a protein which is at least 1% (e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more) shorter than the reference protein. A truncation may further occur at either end (e.g., terminus) of the reference protein (e.g., including the N-or C-terminus) or internal to the protein (e.g., not including either of the terminal residues of the reference protein. In some embodiments, a truncation occurs at the N-terminus of the protein. In some embodiments, a truncation occurs at the C-terminus of the protein. In some embodiments, a truncation occurs between the N-terminus and C-terminus of the protein.

Accordingly, the present disclosure, at least in part, relates to a method of diagnosing a subject for idiopathic normal pressure hydrocephalus (iNPH), wherein the method comprises (a) measuring in a biological sample from the subject a level of Cwh43 protein; and (b) comparing the level of Cwh43 protein in the sample from the subject with a control level of Cwh43 protein. In another aspect, the present disclosure, at least in part, relates to a method of diagnosing a subject for idiopathic normal pressure hydrocephalus (iNPH), wherein the method comprises: (a) analyzing the CWH43 gene in a sample from a subject; and (b) identifying one or more mutations that negatively affect ER export signal of CWH43.

In another aspect, the present disclosure, at least in part, relates to a method of diagnosing a subject for idiopathic normal pressure hydrocephalus (iNPH), wherein the method comprises: (a) identifying a truncated Cwh43 protein in a biological sample obtained from a subject.

Additionally, a subject may also be assessed and/or treated for iNPH by evaluating the CWH43 gene for mutations in the nucleic acid sequence (e.g., DNA, RNA (e.g., mRNA)) encoding the Cwh43 protein. Accordingly, in some aspects, the disclosure relates to a method of treating idiopathic normal pressure hydrocephalus (iNPH) in a subject, comprising: (a) analyzing the CWH43 gene in a sample from a subject; and (b) determining if any mutations in the CWH43 gene in the subject negatively affects the endoplasmic reticulum (ER) export signal of the CWH43 gene product; wherein the subject is administered at least one treatment for iNPH if the CWH43 gene product has at least one mutation negatively affecting the ER export signal of the CWH43 gene product. Various techniques are known in the art for assessing genes, and/or the expression thereof. For example, without limitation, in some embodiments, genes, and/or expression thereof, are assessed (e.g., measured, quantified, evaluated for mutations, evaluated for presence) by any of the following: northern blots, serial analysis of gene expression (SAGE), microarray analysis, polymerase chain reactions (e.g., PCT, RT-PCR, etc.), use of reporter gene, fluorescent in situ hybridization, tiling arrays, RNA-Seq (e.g., high-throughput gene sequencing), indirectly by measuring protein levels as described elsewhere herein, or a combination thereof.

Further, a subject may be assessed and/or treated for iNPH by evaluating the Cwh43 protein structure found in a sample against a control protein. Accordingly, in some aspects, the disclosure relates to a method of treating idiopathic normal pressure hydrocephalus (iNPH) in a subject, comprising: (a) isolating Cwh43 protein from a sample from the subject; and (b) comparing the isolated Cwh43 protein from the sample from the subject with a control Cwh43 protein; wherein the subject is administered at least one treatment for iNPH if the isolated Cwh43 is truncated compared to the control Cwh43 protein.

The terms “biological sample” and “sample,” as may be used interchangeably herein, refer to any sample of a subject, including without limitation, spinal fluid (e.g., cerebra-spinal fluid (CSF)), blood, saliva, cells (e.g., skin cells, tissue cells), bone, cartilage, or generally any other component of the subject or aforementioned examples. In some embodiments, the sample is taken expressly for the purpose of performing the methods or detection of the instant disclosure. In some embodiments, the sample was previously taken from the subject. In some embodiments, the sample was taken, either previously or contemporaneously, for another purpose and is also used in the methods disclosed herein.

The term “mutation,” as may be used herein, refers to a change, alteration, or modification to a nucleotide, or addition or deletion of at least one nucleotide, in a nucleic acid as compared to its wild-type sequence or a change, alteration, or modification to an amino acid, or addition or deletion of at least one amino, in an amino acid sequence (e.g., peptide, polypeptide, protein) as compared to its wild-type sequence. For example, without limitation, mutations may include substitutions, insertions, deletions, or any combination of the same. The terms “wild type” and “native,” as may be used interchangeably herein, are terms of art understood by skilled artisans and mean the typical form of an item, organism, strain, gene, or characteristic as it occurs in nature as distinguished from engineered, mutant, or variant forms. In some embodiments, there at least one mutation. In some embodiments, there are more than one mutation. In some embodiments, where there is more than one mutation, the mutations are distinct (e.g., not of the same type (e.g., substitutions, insertions, deletions)). In some embodiments, where there is more than one mutation, the mutations are the same (e.g., not of the same type (e.g., substitutions, insertions, deletions)). Additionally, in some embodiments, the mutations result in a frameshift. In some embodiments a mutation is a deletion. In some embodiments, a CWH43 gene comprises at least 1 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more) mutation. In some embodiments, a CWH43 gene comprises less than 10,000 (e.g., 5,000; 1,000; 500; 100; 50; 25; 10; 5; 2) mutations. In some embodiments, a CWH43 gene comprises between 1 and 100 mutations. In some embodiments, the CWH43 gene comprises between 1 and 50 mutations. In some embodiments, the CWH43 gene comprises between 1 and 25 mutations. In some embodiments, the CWH43 gene comprises between 1 and 11 mutations.

In some embodiments the one or more mutations results in a homozygous CWH43 deletion. In some embodiments the one or more mutations results in a heterozygous CWH43 deletion. In some embodiments the one or more mutations results in a CWH43 deletion (e.g., a nucleotide deletion) that disrupts the carboxyl terminus (e.g., C-terminus, COOH-terminus) of the Cwh43 protein (e.g., causes a frameshift mutation inserting a stop codon resulting in truncated Cwh43 protein). In some embodiments the one or more mutations results in a heterozygous CWH43 deletion (e.g., a nucleotide deletion) that disrupts the carboxyl terminus of the Cwh43 protein (e.g., causes a frameshift mutation inserting a stop codon resulting in truncated Cwh43 protein). In some embodiments, the one or more mutations are set forth in Table 1. In some embodiments, a mutation is selected from one or more of the following: K669fs, and Leu533Ter. As will be immediately apparent to the skilled artisan, where citing various mutations herein (e.g., K669fs, Leu533Ter), customary notation is used. For example, the first letters preceding a number will represent the amino acid residue of a wild-type variant of a protein to be affected by the mutation, e.g., ‘K,’ ‘Leu,’ which represent the amino acids lysine and leucine, respectively. The letters may be the full name of the amino acid, or their respective three or one letter abbreviations. The following numbers represent the position of the mutation by residue number, in the protein, where the mutation occurs (e.g., 669, 533). The terminal portion of the mutation notation (e.g., remaining letters following the positional numbers), represent the mutation which occurs, e.g., fs, Ter, (or other notation (e.g., amino acid) which may indicate a mutation (e.g., substitution) as is customary mutation notation) which represent a frameshift and termination of the protein, respectively. Frameshifts mutations result from the insertion or deletion of a number of nucleotides in a nucleic acid sequences, which number is not divisible by 3. Due to this property, and the property of codons encoding amino acids in sets of 3, translation is affected by the frameshift. Accordingly, the sequence of proteins resulting from a frameshift (e.g., fs) mutation will typically be longer or shorter than their wild-type counterparts. Accordingly, in instances where a frameshift is indicated, additional information may be included, such as the initial residue of the frameshift (e.g., Asn, N) and/or the number of residues introduced into the sequence (e.g., 23, indicating 23 amino acids were introduced into the sequence).

In some embodiments, the one or more mutations, substitutions, insertions, or deletions results in translation of a truncated gene product (e.g., a truncated Cwh43 protein) or reduced expression of a gene product (e.g., a deletion of a CWH43 gene resulting in reduction of Cwh43 protein translation in a subject). In some embodiments, methods involve detecting one or more proteins, such as one or more gene products of a CWH43 gene (e.g., full-length Cwh43 protein, truncated Cwh43 protein, etc.). In some embodiments, methods further comprise administering to the subject a therapeutic agent (e.g., a nucleic acid, peptide, small molecule, or any combination thereof) or performing a surgical procedure on a subject (e.g., CSF drainage).

In some embodiments, a mutation to the CWH43 gene and or Cwh43 protein, negatively affects export of the protein from the endoplasmic reticulum (ER). Export of proteins from the ER depends, at least in part, on the interaction of a signal motif (ER export signal) on the cargo protein by the export machinery. ER export signals are well known in the art and will be readily appreciated by the skilled artisan. In some embodiments, a mutation negatively affects the ER export signal of a Cwh43 protein. As used herein, the term “negatively,” in the context of an affect, refers to a degraded, impeded, or less efficacious state or activity level as compared to the activity of its wild-type counterpart. For example, a negatively affected ER export signal may be the deletion of an ER export signal or mutation thereof, such that the signal does not effectuate as much, or the same level of, export of the protein (e.g., Cwh43) from the ER as when the ER export signal is not deleted, mutated, or the activity thereof attenuated (e.g., by inclusion of counteracting signals, signals with inhibitory or opposite activity, or other compositions which effectuate similar outcomes). In some embodiments, a mutation to the CWH43 gene comprises a deletion of a sequence comprising a ten base pair sequence of SEQ ID NO: 6. In some embodiments, an ER export signal may be deleted because the protein was truncated prior to the translation of the ER export signal. In some embodiments, a mutation negatively affects the ER export signal by causing a truncation of the protein at, or near, residue 533. In some embodiments, the ER export signal is negatively affected by the introduction of at least one ER retention signal. In some embodiments, the ER retention signal comprises the amino acid sequence of SEQ ID NO: 4. In some embodiments, the ER export signal is negatively affected by the introduction of at least one ER retrieval signal. In some embodiments, the ER retrieval signal comprises the amino acid sequence of SEQ ID NO: 7. ER retention and retrieval signals are generally known to be signals (e.g., peptides, polypeptides, proteins) which allows the ER to retain proteins in the ER or influence their localization to the ER.

In some aspects, the disclosure relates to a method of treating idiopathic normal pressure hydrocephalus (iNPH) in a subject, comprising: (a) measuring in a sample from the subject the level of Cwh43 protein; and (b) comparing the level of Cwh43 protein found in the sample from the subject with a control level of Cwh43 protein; wherein the subject is administered at least one treatment for iNPH if the level of Cwh43 is lower than control level of Cwh43 protein.

Evaluation of a CWH43 gene may be performed in any manner known in the art. For example, nucleic acids in a sample from a subject may be sequences for evaluation of the CWH43 gene. In some embodiments, sequencing may be performed on the whole genome of a subject. In some embodiments, the RNA of a subject is sequenced. In some embodiments, the exons are the focus of the sequencing. In some embodiments, sequencing comprises sequencing the exome of a subject.

Measurement of proteins in samples obtained from a subject is readily understood by the skilled artisan and may be accomplished by any method known in the art. For example, it is well known that protein quantification (e.g., detection) may be performed by enzymatic activity, antibody conjugation, spectroscopic analysis (e.g., colorimetry), protein labeling (e.g., radiolabeling, fluorescent tagging) and quantification, isolation (e.g., chromatography (e.g., ion exchange, size exclusion, gel filtration, affinity chromatography), gel electrophoresis, high performance liquid chromatography (HPLC), liquid chromatography mass spectroscopy (LC/MS), enzyme linked immunosorbent assays (ELISA), immunoprecipitation, immunoelectrophoresis, western blots, protein staining, and/or combinations of the aforementioned. Accordingly, the skilled artisan will readily be able to ascertain an appropriate method to determine protein levels in practicing the instant methods. In some embodiments, proteins in the methods of the present disclosure are determined by any one of the following: enzymatic activity, antibody conjugation, spectroscopic analysis (e.g., colorimetry), protein labeling (e.g., radiolabeling, fluorescent tagging) and quantification, isolation (e.g., chromatography (e.g., ion exchange, size exclusion, gel filtration, affinity chromatography), gel electrophoresis, high performance liquid chromatography (HPLC), liquid chromatography mass spectroscopy (LC/MS), enzyme linked immunosorbent assays (ELISA), immunoprecipitation, immunoelectrophoresis, western blots, protein staining, and/or a combination thereof. In some embodiments, a protein is measured (e.g., assessed, quantified) by western blot.

In some embodiments, a protein is measured by use of an antibody. In some embodiments, the antibody is used in an assay (e.g., antibody-based assay, immunoassay). As used herein, an antibody-based assay is an assay in which an antibody to a target of interest (e.g., protein to be measured (e.g., Cwh43)) is conjugated to a detectable tag (e.g., reporter, detectable tag), which upon the addition of its substrate, the enzyme catalyzes the activation or incorporation of the detectable tag (e.g., production of a detectable signal). The detectable tag may then be assessed by exploiting a known property thereof, e.g., color, magnetism, affinity, chemical property, etc. Such assays and techniques are well known in the art and will readily be understood and appreciated by the skilled artisan. In some embodiments, the antibody comprises a moiety, or detectable tag.

In some embodiments, the protein measured is a protein in a sample from a subject. In some embodiments, the protein measured is a control level protein. As used herein, the term “control” refers to a level or value (e.g., value as quantified, measurement value equating to a level of a protein) of a protein, used as a reference value to establish a benchmark against which the measured value from a subject may be compared. Use of controls is a technique well established throughout the art and will readily be understood by the skilled artisan. Further, the skilled artisan will immediately understand and be able to ascertain a control level without undue experimentation.

In some embodiments, a Cwh43 protein measured in the subject is a wild-type Cwh43 protein. In some embodiments, a Cwh43 protein is a human Cwh43 protein. In some embodiments, a Cwh43 protein comprises a sequence with at least 70% identity to SEQ ID NO: 2. In some embodiments, a Cwh43 protein comprises a sequence of SEQ ID NO: 2. The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%,at least 96%, at least 96.5%,at least 97%, at least 97.5%,at least 98%, at least 98.5%,at least 99%, at least 99.5%, at least 99.6%, at least 99.7%, at least 99.8%, at least 99.9% identity) and all increments thereof (e.g., tenths of a percent (e.g., 0.1%), hundredths of a percent (e.g., 0.01%), etc.).

In some embodiments a Cwh43 protein is a non-wild-type Cwh43 protein. In some embodiments, a Cwh43 protein results from a mutation in the nucleic acid encoding the Cwh43 protein. In some embodiments, a Cwh43 protein comprises a truncation. In some embodiments, a Cwh43 protein is truncated at its carboxyl terminus (C-terminus). In some embodiments, a truncation occurs between residues 525 and 541 (e.g., residue 525, 526, 527, 528, 529, 530, 531, 532, 533, 534, 535, 536, 537, 538, 539, 540, or 540) of a Cwh43 protein. In some embodiments, a truncation occurs between residues 530 and 536 of a Cwh43 protein. In some embodiments, a truncation occurs between residues 532 and 534 of a Cwh43 protein. In some embodiments, a truncation occurs at, or around residue 533 of a Cwh43 protein. In some embodiments, a truncation occurs at residue 533 of a Cwh43 protein. In some embodiments, a truncation occurs at, or near a leucine residue. In some embodiments, a truncation occurs within 1 amino acid of a leucine residue. In some embodiments, a truncation occurs at a leucine residue.

In some embodiments, a Cwh43 protein comprises a frameshift mutation. In some embodiments, a Cwh43 protein has a frameshift in its carboxyl terminus (C-terminus). In some embodiments, a frameshift occurs between residues 690 and the terminal residue of a Cwh43 protein. In some embodiments, a frameshift occurs between residues 693 and 699 (e.g., residues 693, 694, 695, 696, 697, 698, or 699) of a Cwh43 protein. In some embodiments, a frameshift occurs between residues 695 and 697 of a Cwh43 protein. In some embodiments, a frameshift occurs at, or around residue 696 of a Cwh43 protein. In some embodiments, a frameshift occurs at residue 696 of a Cwh43 protein. In some embodiments, a frameshift occurs at, or near a lysine residue. In some embodiments, a frameshift occurs within 1 amino acid of a lysine residue. In some embodiments, a frameshift occurs at a lysine residue. In some embodiments, the frameshift introduces at least 1 amino acid. In some embodiments, the frameshift introduces at least 5 amino acids. In some embodiments, the frameshift introduces at least 10 amino acids. In some embodiments, the frameshift introduces at least 15 amino acids. In some embodiments, the frameshift introduces at least 20 amino acids. In some embodiments, the frameshift introduces at least 25 amino acids. In some embodiments, the frameshift introduces at least 23 amino acids. In some embodiments, the frameshift introduces amino acids which comprise an ER retention signal. In some embodiments, the ER retention signal comprises SEQ ID NO: 4. In some embodiments, the frameshift mutation comprises an insertion of 22, 23, or 24 amino acids into the Cwh43 protein and comprises an ER retention signal of SEQ ID NO: 4. In some embodiments, the frameshift occurs at residue 696 and inserts a sequence comprising SEQ ID NO: 5.

In some embodiments, the Cwh43 protein level measured in the subject is compared to a control level of a protein. In some embodiments, the control level may be a control level of a Cwh43 protein. In some embodiments, the control level may be an average level of Cwh43 protein levels found in at least one healthy individual of the same species as the subject. In some embodiments, the control level may be an average level of Cwh43 protein levels found in more than one healthy individual of the same species as the subject. In some embodiments, the control level may be an average level of Cwh43 protein levels found in at least one healthy individual, but less than 100,000 healthy individuals, of the same species as the subject. In some embodiments, the control level may be an average level of Cwh43 protein levels found in at least one healthy individual, but less than 10,000 healthy individuals, of the same species as the subject. In some embodiments, the control level may be an average level of Cwh43 protein levels found in at least one healthy individual, but less than 1,000 healthy individuals, of the same species as the subject. In some embodiments, the control level may be an average level of Cwh43 protein levels found in at least one healthy individual, but less than 100 healthy individuals, of the same species as the subject. In some embodiments, the control level may be an average level of Cwh43 protein levels found in at least one healthy individual, but less than 10 healthy individuals, of the same species as the subject. In some embodiments, the control level is obtained from a database. The control level may be evaluated and measured in any of the methods mentioned herein for establishing and measuring levels of Cwh43 protein levels in a subject or sample. In some embodiments, a control level is a Cwh43 protein. In some embodiments, a Cwh43 protein is a wild-type Cwh43 protein. In some embodiments, the Cwh43 protein is a human Cwh43 protein. In some embodiments, the Cwh43 protein comprises a sequence with at least 70% identity to SEQ ID NO: 2. In some embodiments, the Cwh43 protein comprises a sequence of SEQ ID NO: 2.

In some embodiments, a measured Cwh43 protein level from a subject is compared to a Cwh43 protein control level. In doing so, the two values are compared to assess which value is greater or to establish a relationship between the two values (e.g., ratio). In some embodiments, the two values are compared such that the difference is quantified. In some embodiments, the two values are compared such that a ratio is established. Calculation and/or establishment of a relationship (e.g., calculating the difference, calculating a ratio) refers to analyzing the values to assess the amount of a Cwh43 protein level in a sample from a subject as compared to that of the control value, thereby assessing if the level is higher or lower (or equivalent) than the value of the control value. In some embodiments, a difference between the measured value of Cwh43 in a sample from a subject and a control value, wherein the control value is greater than the measured value, indicates that there is less Cwh43 protein in the sample from the subject, and that the subject has less expressed Cwh43 protein as compared to the control. In some embodiments, a difference between the measured value of Cwh43 in a sample from a subject and a control level, wherein the control value is less than the measured value, indicates that there is more Cwh43 protein in the sample from the subject, and that the subject has more expressed Cwh43 protein as compared to the control.

In some embodiments, a ratio between the measured value of Cwh43 in a sample from a subject and a control level is established with the control level in the denominator, wherein a ratio of less than 1 (e.g., 0.9, 0.8, 0.7, 0.6, 0.5, 0.4, 0.3, 0.2, 0.1, or less), indicates that there is less Cwh43 protein in the sample from the subject, and that the subject has less expressed Cwh43 protein as compared to the control (and conversely a ratio greater than 1 indicates more expressed Cwh43 protein as compared to the control). In some embodiments, a ratio between the measured value of Cwh43 in a sample from a subject and a control level is established with the control level in the denominator, wherein a ratio of between 1 and 0.001, indicates that there is less Cwh43 protein in the sample from the subject, and that the subject has less expressed Cwh43 protein as compared to the control (and conversely a ratio greater than 1 indicates more expressed Cwh43 protein as compared to the control). In some embodiments, a ratio between the measured value of Cwh43 in a sample from a subject and a control level is established with the control level in the denominator, wherein a ratio of between 1 and 0.01, indicates that there is less Cwh43 protein in the sample from the subject, and that the subject has less expressed Cwh43 protein as compared to the control (and conversely a ratio greater than 1 indicates more expressed Cwh43 protein as compared to the control). In some embodiments, a ratio between the measured value of Cwh43 in a sample from a subject and a control level is established with the control level in the denominator, wherein a ratio of between 1 and 0.1, indicates that there is less Cwh43 protein in the sample from the subject, and that the subject has less expressed Cwh43 protein as compared to the control (and conversely a ratio greater than 1 indicates more expressed Cwh43 protein as compared to the control).

In some embodiments, a ratio between the measured value of Cwh43 in a sample from a subject and a control level is established with the control level in the numerator, wherein a ratio of greater than 1 (e.g., 1; 2; 5, 10; 25; 50; 100; 500; 1,000; or more), indicates that there is less Cwh43 protein in the sample from the subject, and that the subject has less expressed Cwh43 protein as compared to the control (and conversely a ratio less than 1 indicates more expressed Cwh43 protein as compared to the control). In some embodiments, a ratio between the measured value of Cwh43 in a sample from a subject and a control level is established with the control level in the numerator, wherein a ratio of between 1 and 1,000, indicates that there is less Cwh43 protein in the sample from the subject, and that the subject has less expressed Cwh43 protein as compared to the control (and conversely a ratio less than 1 indicates more expressed Cwh43 protein as compared to the control). In some embodiments, a ratio between the measured value of Cwh43 in a sample from a subject and a control level is established with the control level in the numerator, wherein a ratio of between 1 and 100, indicates that there is less Cwh43 protein in the sample from the subject, and that the subject has less expressed Cwh43 protein as compared to the control (and conversely a ratio less than 1 indicates more expressed Cwh43 protein as compared to the control). In some embodiments, a ratio between the measured value of Cwh43 in a sample from a subject and a control level is established with the control level in the numerator, wherein a ratio of between 1 and 10, indicates that there is less Cwh43 protein in the sample from the subject, and that the subject has less expressed Cwh43 protein as compared to the control (and conversely a ratio less than 1 indicates more expressed Cwh43 protein as compared to the control).

In some embodiments, where there is less Cwh43 protein expressed in the sample (e.g., as measured against a control level (e.g., the methods as disclosed herein)), a treatment is administered to the subject.

In some embodiments, any of the methods of the disclosure are performed in conjunction with at least one additional method for determining the risk of iNPH in the subject. In some embodiments, the at least one additional method comprises a method selected from: evaluation for symmetric gait disturbances, evaluation for dementia, evaluation for incontinence, and a negative determination of other causes of hydrocephalus. In some embodiments, the at least one additional method comprises evaluation for symmetric gait disturbances. In some embodiments, the at least one additional method comprises evaluation for dementia. In some embodiments, the at least one additional method comprises evaluation for incontinence. In some embodiments, the at least one additional method comprises a negative determination of other causes of hydrocephalus.

### EXAMPLES

### Example 1: Deletions in CWH43 Cause Sporadic Idiopathic Normal Pressure Hydrocephalus

### Introduction

Idiopathic normal pressure hydrocephalus (iNPH) is a neurological disorder of aging that is characterized by enlarged cerebral ventricles, gait difficulty, incontinence, and dementia. Idiopathic normal pressure hydrocephalus affects an estimated 700,000 Americans, but most remain undiagnosed because many practitioners are unfamiliar with the disease or doubt its existence.

### SUMMARY

Whole-exome sequencing of DNA obtained from 53 unrelated iNPH patients was performed. The patients were enrolled and their DNA was analyzed in three independent cohorts. Single nucleotide variations (SNVs) and short insertions and deletions (indels) that were statistically over-represented among iNPH patients, present in at least two of the three cohorts, and predicted to adversely affect protein function were identified. Deletions affecting the most frequently altered gene were studied further using genetically engineered mice and human cell lines. Magnetic resonance imaging, immunohistochemistry, electron microscopy, gait analysis, and other methods were used to examine the effects of iNPH-associated genetic alterations in mice and in cultured cells.

A heterozygous deletion in CWH43 was observed in four iNPH patients and was enriched 6.6-fold among iNPH patients when compared to the general population (P<0.0002, X² Test). A second heterozygous CWH43 deletion was identified in four additional patients and was enriched 2.7-fold (P<0.0406, X² Test). In yeast, Cwh43 incorporates ceramide into the lipid anchor of glycosylphosphatidylinositol (GPI) anchored proteins, thereby regulating their membrane localization. Here, it is shown that Cwh43 regulates the membrane localization of GPI-anchored proteins in mammalian cells, and both of the iNPH-associated CWH43 deletions disrupt this function. In the mouse brain, Cwh43 expression is high in ciliated ependymal and choroid plexus cells. Mice heterozygous for one of the CWH43 deletions appeared grossly normal but displayed enlarged ventricles, gait and balance abnormalities, decreased numbers of ependymal cilia, and decreased localization of GPI-anchored proteins to the apical surfaces of choroid plexus and ependymal cells.

Approximately 15% of patients with sporadic iNPH harbor heterozygous loss of function deletions in CWH43. Mice harboring iNPH-associated CWH43 deletions develop communicating hydrocephalus, gait dysfunction, and choroid plexus, and ependymal cell abnormalities. The findings provide new mechanistic insights into the origins of iNPH and demonstrate that it represents a distinct disease entity.

### Discussion

Idiopathic normal pressure hydrocephalus is a neurological disorder of aging that is characterized by enlarged cerebral ventricles, gait difficulty, incontinence, and dementia. Because the symptoms can be improved by CSF drainage, iNPH is typically classified as a reversible dementia. Idiopathic normal pressure hydrocephalus affects about 700,000 Americans and occurs almost exclusively after the age of 60 (the average age at diagnosis is about 75 years). Researchers have estimated that 1.4% to 2.9% of the population over the age of 65, 5.6% of the population over the age of 75, and nearly 1 in 7 nursing home residents has iNPH. Unfortunately, most of these patients are misdiagnosed or undiagnosed, in part because the symptoms of iNPH resemble the symptoms of other neurological disorders such as Parkinson's Disease or Alzheimer's Disease, and in part because many health care providers and the lay public are unfamiliar with this disorder. Importantly, not all patients who present with ventriculomegaly, gait difficulty, incontinence, and dementia improve after CSF drainage, such that a diagnosis of shunt-responsive iNPH is only confirmed if the symptoms improve after CSF drainage.

Ventricular CSF stasis, abnormal cerebrovascular blood flow and reactivity, and increased amplitude of intracranial pressure waves have all been observed in iNPH patients. Associations between iNPH and hypertension, hypercholesterolemia, diabetes, and alcohol consumption have been reported, but the physiological mechanisms underlying these associations are not known. A recent single nucleotide polymorphism study reported intronic copy number loss in the SFMBT gene in 26% of iNPH patients compared to 4.2% of the general population, but the significance of this finding was not determined. This example describes identification of genetic abnormalities associated with sporadic shunt-responsive iNPH.

### Methods

Fifty-three unrelated patients who presented with ventricular enlargement, gait difficulty, incontinence, and/or dementia underwent an evaluation that included a history, neurological examination and trial of lumbar CSF drainage. Patients were consented for the study prior to CSF drainage, and only those who improved after ventriculoperitoneal shunt placement were included for whole exome analysis.

The 53 patients with shunt-responsive iNPH were enrolled and analyzed in three separate cohorts (n=20, n=12, and n=21). Genomic DNA was isolated from whole blood and submitted in three independent batches for whole exome sequencing (50× coverage, 150 base pair (bp) paired end sequencing, Illumina HiSeq 2000). Single nucleotide variants (SNVs) and insertions/deletions (indels) were identified (Human Genome build GRCh37, bwa-mem, Genome Analysis Toolkit HaplotypeCaller). Genetic alterations with a frequency greater than 1% in the 1000 Genomes Project database or the ExAC database were initially excluded. The minor allele frequency (MAF) of each mutation in the study group was compared to that in the general population (i.e., the combined MAF across all ethnic groups, ExAC database), and statistical enrichment among iNPH patients was calculated using the two tailed X² test with and without the Yates correction. Four computer prediction algorithms (SIFT, Provean, Mutation Tester, and Polyphen 2) were used to predict the effect of each mutation on protein function. Genes with three or more mutations that were predicted to be damaging by at least two of the four computer prediction algorithms were selected for further study. Manual curation of whole exome sequencing data for CWH43, a gene that harbored the most recurrent damaging mutation, revealed another recurrent damaging deletion with a MAF of 0.0142 in the general population. This deletion resulted in a frameshift and truncation of the encoded protein, and was thus included in the study. Genetic alterations were confirmed using polymerase chain reaction (PCR) and Sanger sequencing.

In situ mRNA hybridization images for CWH43 were obtained from a public database (Allen Brain Atlas). Cryostat sections of mouse brains were prepared and stained for fluorescence immunohistochemistry using antibodies directed against Cwh43, CD59, folate receptor alpha, or acetylated a-tubulin. Nuclei were counterstained using 4′,6-diamidino-2-phenylindole (DAPI).

Ventricular ependymal cells from newborn wild-type mice were dissociated and cultured in medium containing 2% serum. The cells were then fixed in paraformaldehyde, stained using antibodies against Cwh43 and acetylated a-tubulin (to visualize cilia), and imaged using fluorescence confocal microscopy.

Human HeLa cells harboring the CWH43 deletion (4:49034669 CA/C; Leu533Ter) were generated using the CRISPR/Cas9 method. The mutation was confirmed by DNA sequencing, and loss of full length Cwh43 protein expression was confirmed by Western blot. Expression plasmids for wild-type human CWH43 and human CWH43 harboring either the iNPH-associated deletion (4:49063892 CA/C; Lys696AsnfsTer23) or (4:49034669 CA/C; Leu533Ter) were generated using site specific mutagenesis. Expression plasmids encoding Green Fluorescent Protein (GFP) fused to the N-terminus of these CWH43 variants were also generated. HeLa cell lines stably expressing each fusion protein were then generated by transient transfection followed by antibiotic selection.

C57b16 mice harboring a Met533Ter mutation (coinciding to the human CWH43 deletion 4:49034669 CA/C; Leu533Ter) were generated using a CRISPR/Cas9 approach and bred to generate heterozygous (CWH43^{WT/M533}) and homozygous (CWH43^{M533/M533}) animals. To control for CRISPR/Cas9 off target effects, a second C57b16 mouse line was independently generated, harboring one mutant Met533Ter allele and one allele containing a 10 base pair deletion (ACCAGCCATA (SEQ ID NO: 6)) in CWH43 that generates a stop codon at A530 (CWH43^{M533/A530}). Both CWH43 mutant mouse lines were studied for comparison.

T2-weighted MRI images of the brains of CWH43 mutant mice were obtained, and ventricular volume was calculated using ImageJ and a custom automated computer algorithm. In some cases, mouse brains were harvested and the ventricular surface was fixed for examination using immunohistochemistry or scanning electron microscopy. Color-coded spatial analysis of gait in mice was performed using a place mat and quantitative measurement of stride length, stance, and sway. Evaluation of strength, balance, and coordination in mice was performed using the rotarod performance test. Statistical significance for laboratory studies was calculated using the two tailed unpaired t-test with a significance threshold of P<0.05.

### Results

### Patient Characteristics

Whole exome sequencing of DNA obtained from 53 patients with shunt-responsive iNPH was performed in three independent cohorts (Table 1). Collectively, there were 29 females and 24 males. The median age was 75 years (range 65-89 years). All of the patients had enlarged cerebral ventricles and gait difficulty. Urinary incontinence and cognitive impairment were present in 79% and 83% of the patients, respectively.

TABLE 1
iNPH Patient Characteristics

| | | | Sx | | | | | CWH43 |
|---|---|---|---|---|---|---|---|---|
| Cohort | Age | Sex | Duration | Gait | Incontinence | Dementia | Improvement | Alteration |
| I | 76 | F | NA | ** | *** | ** | ** | K669fs |
| I | 79 | M | NA | ** | * | None | * | |
| I | 84 | F | 2 | *** | ** | *** | * | |
| I | 80 | F | 2 | ** | ** | *** | * | |
| I | 80 | F | 5 | *** | ** | ** | *** | Leu533Ter |
| I | 68 | M | NA | *** | None | None | ** | |
| I | 76 | F | 2 | ** | * | *** | * | |
| I | 74 | M | 4 | *** | * | * | *** | K669fs |
| I | 84 | F | 2 | *** | ** | None | ** | |
| I | 77 | M | 6 | ** | None | * | * | |
| I | 65 | F | 4 | *** | *** | ** | *** | K669fs |
| I | 76 | F | 2 | * | * | * | ** | |
| I | 69 | M | 2 | *** | * | ** | ** | Leu533Ter |
| I | 67 | M | 1 | ** | ** | ** | *** | |
| I | 76 | M | NA | ** | *** | ** | ** | |
| I | 77 | F | 2 | *** | ** | ** | ** | |
| I | 70 | F | 2 | ** | ** | ** | ** | |
| I | 68 | M | 3 | *** | ** | ** | *** | Leu533Ter |
| I | 89 | M | 10 | *** | *** | ** | *** | |
| I | 81 | M | 1 | *** | *** | ** | *** | |
| II | 75 | M | 2 | * | ** | * | ** | |
| II | 76 | F | 2 | *** | ** | ** | *** | |
| II | 81 | F | 1 | *** | *** | ** | *** | |
| II | 81 | F | 1 | *** | ** | ** | ** | |
| II | 86 | M | 3 | *** | ** | ** | ** | |
| II | 75 | F | NA | *** | ** | ** | * | |
| II | 81 | F | 1 | *** | None | * | * | |
| II | 76 | M | 2 | *** | ** | ** | * | |
| II | 84 | F | 3 | *** | * | * | * | K669fs |
| II | 73 | F | 4 | *** | *** | ** | ** | |
| II | 70 | M | 2 | ** | *** | *** | * | |
| II | 75 | F | 2 | ** | ** | ** | ** | |
| III | 77 | M | 20 | ** | None | None | ** | |
| III | 72 | F | 0.5 | ** | *** | *** | * | |
| III | 78 | M | 1 | ** | * | * | ** | |
| III | 73 | M | NA | ** | None | None | ** | |
| III | 70 | F | 2 | ** | * | * | *** | |
| III | 78 | M | 2 | ** | None | * | ** | |
| III | 75 | F | 0.5 | ** | ** | * | ** | |
| III | 68 | F | 3 | ** | ** | * | * | |
| III | 77 | M | 3 | *** | None | None | ** | |
| III | 75 | M | 2 | ** | None | ** | ** | |
| III | 70 | F | 2 | ** | ** | None | *** | |
| III | 68 | M | 2 | ** | ** | ** | *** | |
| III | 76 | M | NA | *** | *** | ** | *** | |
| III | 75 | F | 2 | *** | ** | ** | ** | |
| III | 68 | M | 1 | ** | ** | ** | ** | Leu533Ter |
| III | 72 | F | 1 | * | * | None | *** | |
| III | 72 | F | 1 | * | None | None | * | |
| III | 81 | F | NA | *** | ** | *** | ** | |
| III | 74 | M | 2 | *** | None | ** | *** | |
| III | 79 | F | 1 | *** | *** | *** | *** | |
| III | 70 | F | 0.5 | *** | * | * | * | |

NA—data not available
None—Symptom not present
“Ter” refers to a termination codon
“fr” refers to a frameshift mutation
*—Mild
**—Moderate
***—Major

### Recurrent iNPH-Associated Deletions

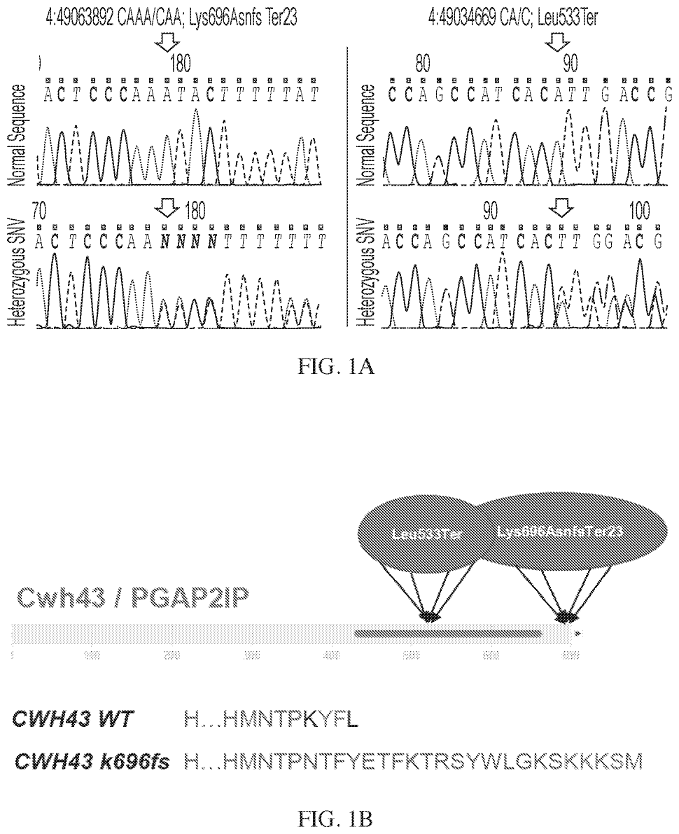

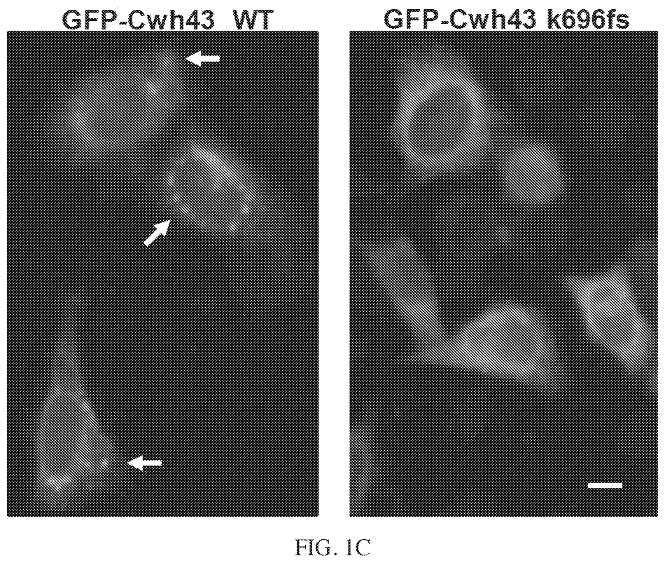

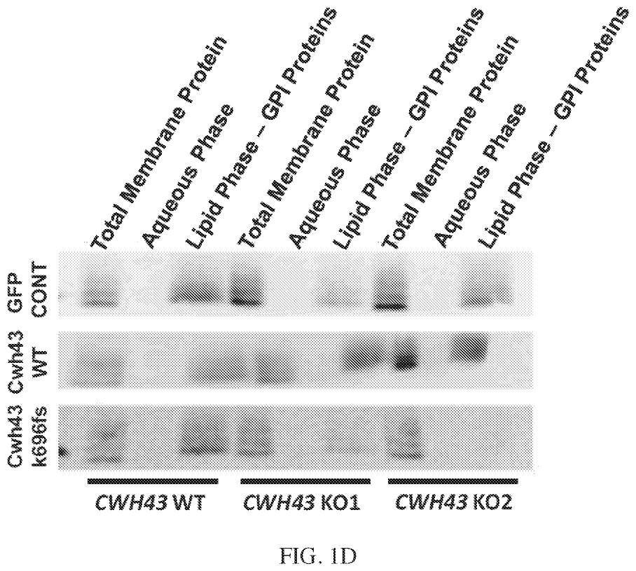

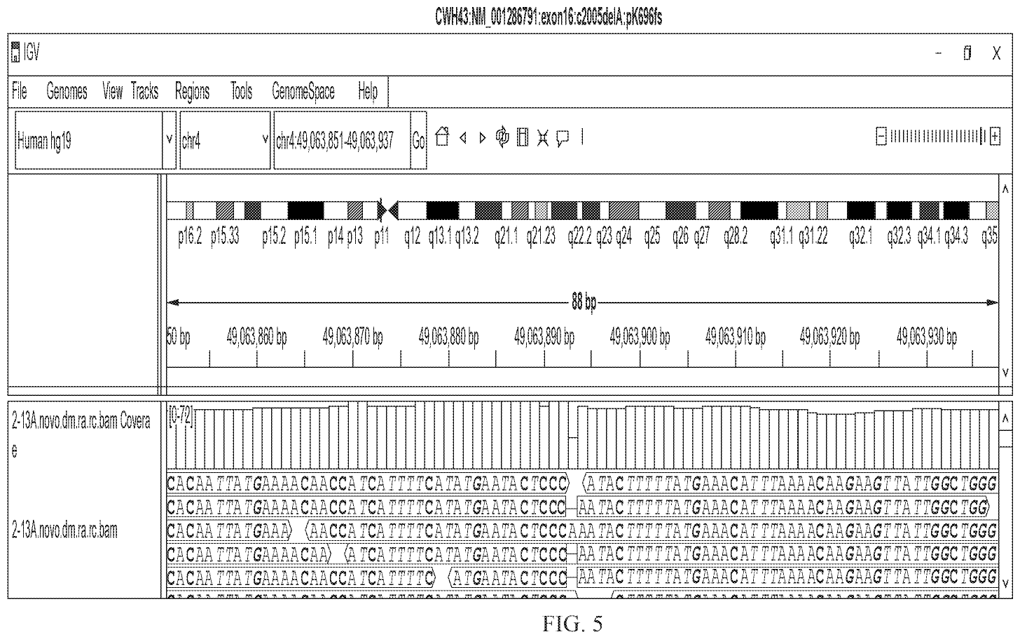

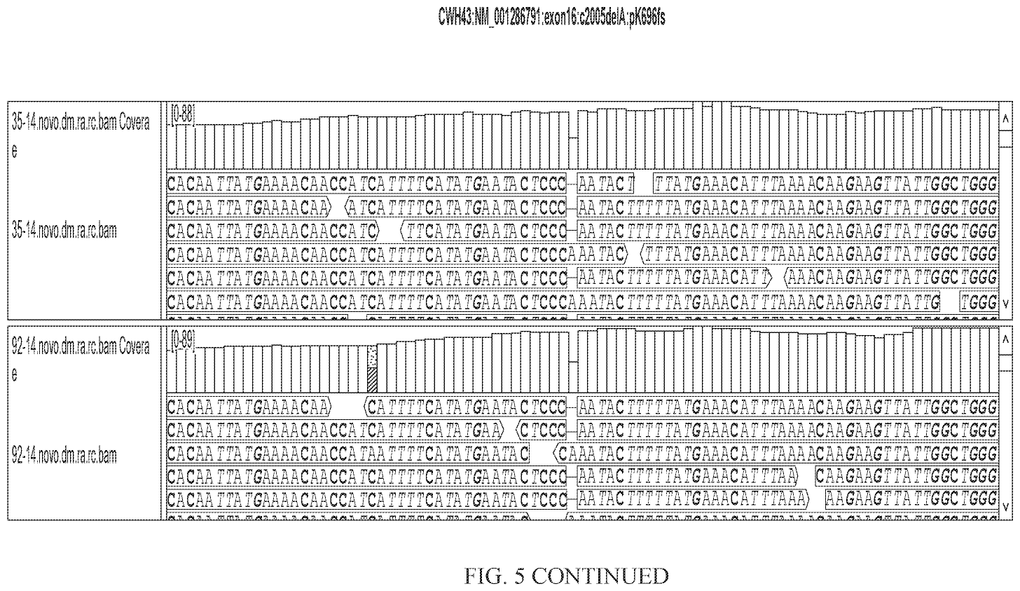

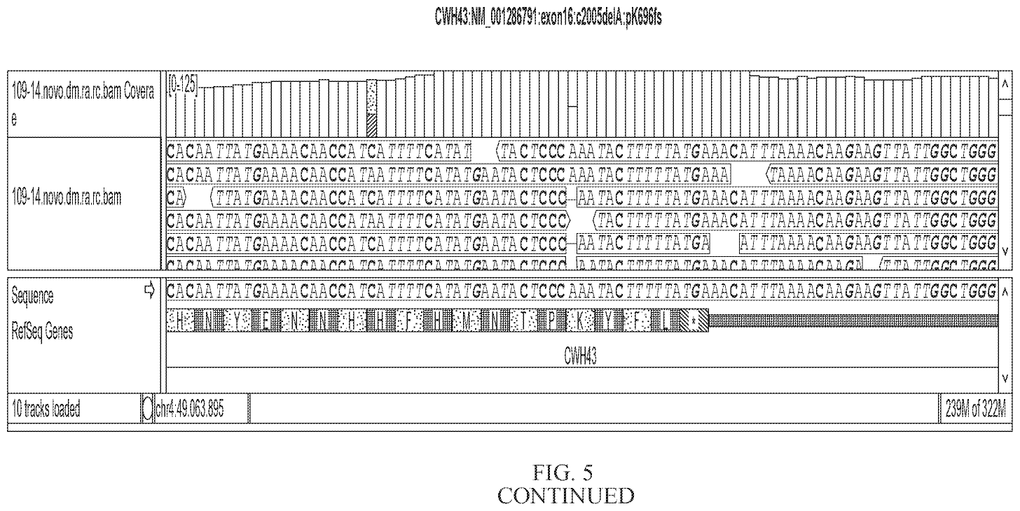

Analysis of sequencing data identified 4 of 53 patients with the same deletion in CWH43 (FIG. 5). The presence of the mutation was confirmed in each case by Sanger sequencing of DNA (FIG. 1A). The mutation (4:49063892 CA/C; Lys696AsnfsTer23) has a minor allele frequency (MAF) of 0.0057 in the general population and 0.0377 among iNPH patients;), a 6.6 fold increase (P<0.0001, X² Test; P<0.0002, X² Test with Yates correction). This deletion, which was heterozygous in each patient and observed in 2 of 3 independent cohorts, leads to a frameshift that alters the carboxyl terminus of Cwh43.

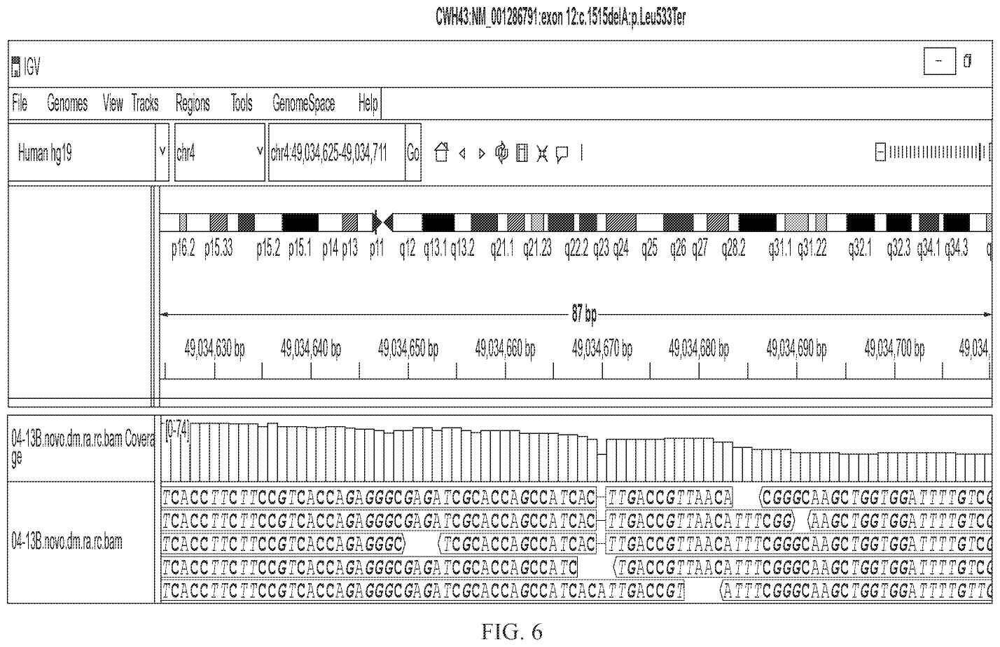

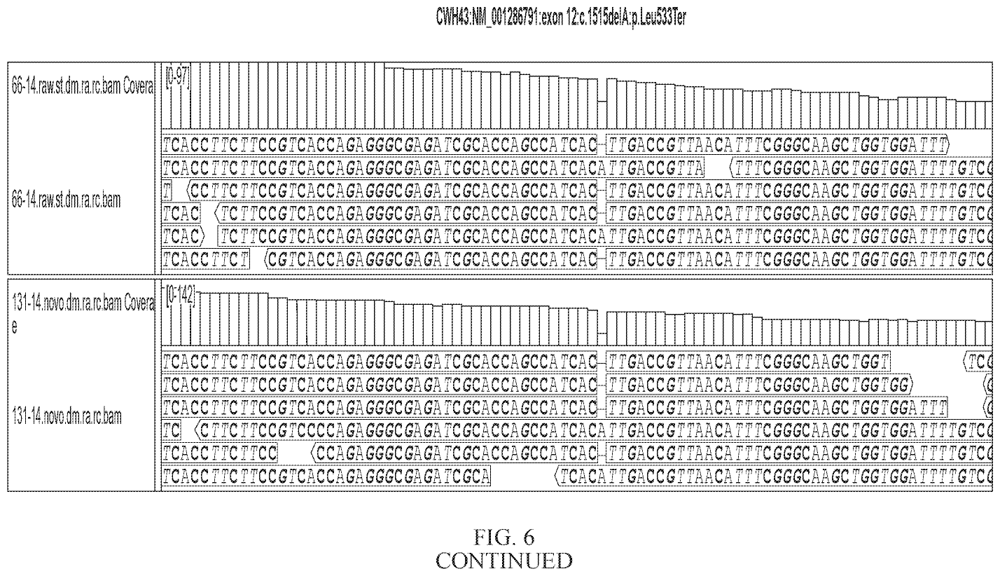

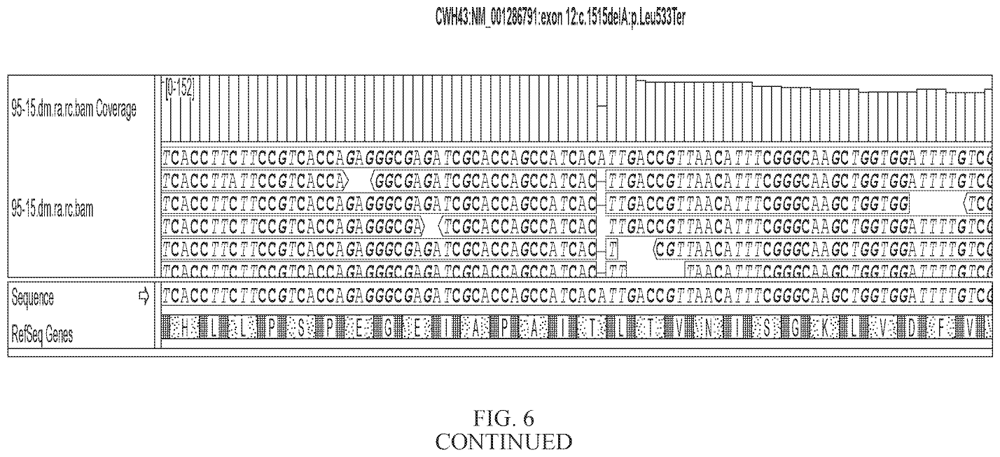

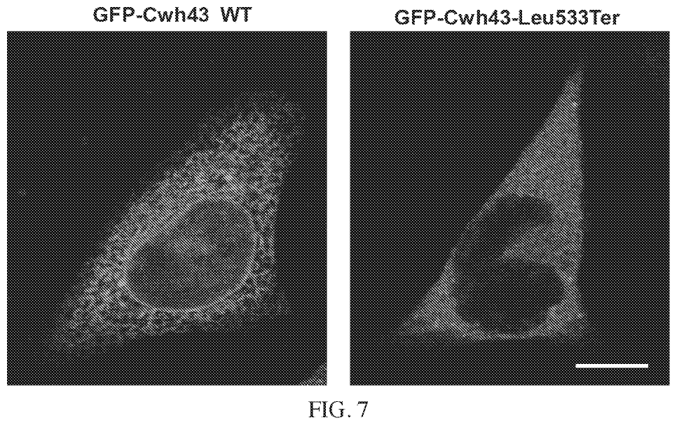

Manual examination of sequencing data identified 4 additional patients harboring a different damaging CWH43 deletion (4:49034669 CA/C; Leu533Ter) (FIG. 6). This CWH43 deletion generates a frameshift that causes premature termination of the Cwh43 protein (FIG. 1A). This second deletion was filtered out during the original analysis because its MAF exceeds 0.01 in the general population. This CWH43 deletion has a MAF of 0.0142 in the general population and 0.0377 in the iNPH cohort, and thus occurred with a 2.7-fold increased frequency (P<0.0406, X² Test; P<0.1016, X² Test with Yates correction). This second CWH43 deletion was also heterozygous in each patient and observed in 2 of 3 independent cohorts.

Taken together, 8 of 53 iNPH patients (15%) carried a heterozygous CWH43 deletion that disrupts the carboxyl terminus of the Cwh43 protein. CWH43 deletions were identified in all 3 independent cohorts. All of the 8 patients presented with gait difficulty, incontinence and cognitive impairment that improved after CSF drainage. Three of these patients had a family history of iNPH or gait difficulty, with one patient describing 3 first degree relatives who had been diagnosed with iNPH.

### Effect of CWH43 Deletions on Cwh43 Function

Yeast Cwh43 (Cell wall biogenesis protein 43 C-terminal homolog) is a transmembrane protein that incorporates ceramide into the glycosylphosphatidylinositol (GPI) anchor that attaches certain proteins to the cell membrane. Incorporation of ceramide into the lipid anchor of GPI-anchored proteins by Cwh43 regulates the membrane localization of this class of proteins in yeast. Although the function of Cwh43 in multicellular organisms is not known, a lipid remodeling domain is predicted to reside near the carboxyl terminus (FIG. 1B). The recurrent iNPH-associated CWH43 mutation (Lys696AsnfsTer23) causes a frameshift that eliminates the last four amino acids of Cwh43 and replaces it with a novel 23 amino acid sequence. This removes an endoplasmic reticulum (ER) export signal (YF) and adds an ER retrieval signal (KKXX (SEQ ID NO: 7), FIG. 1B). Overexpression of a wild-type human GFP-Cwh43 fusion protein or a mutant GFP-Cwh43-K696fs protein in HeLa cells confirmed that wild-type Cwh43-GFP is associated with intracellular vesicles and the Golgi (where modification of the lipid anchor is thought to occur), while GFP-Cwh43-K696fs is more diffusely distributed (FIG. 1C).

The other recurrent CWH43 deletion creates a stop codon at Leu533, thereby generating a truncated Cwh43 protein lacking the C-terminal ER export signal and the putative lipid remodeling domain. Overexpression of human GFP-Cwh43-Leu533Ter in HeLa cells confirmed that the mutant protein shows decreased association with the Golgi and intracellular vesicles, and is diffusely distributed throughout the cytoplasm.

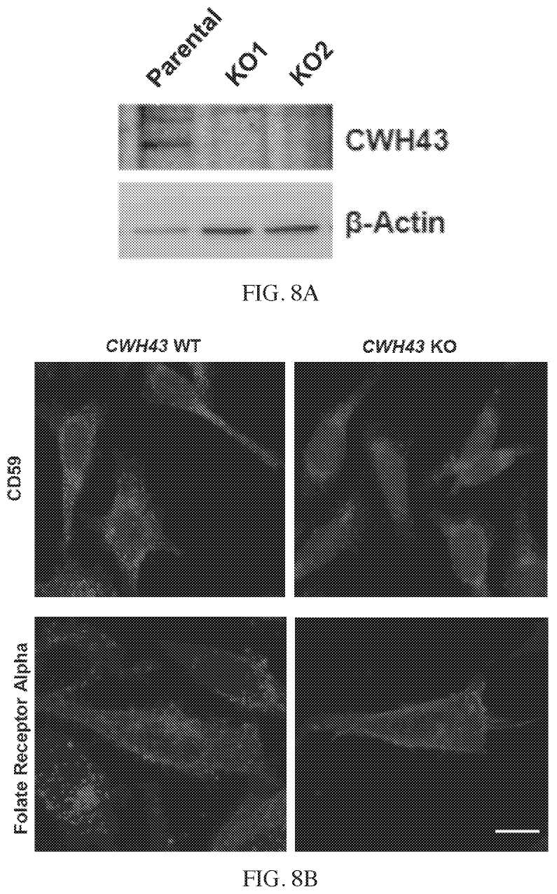

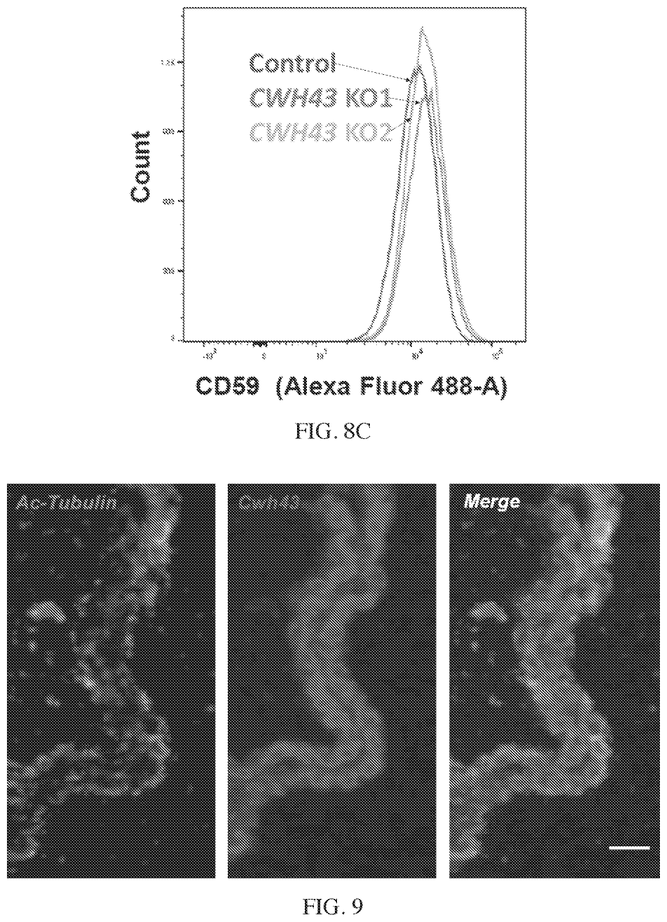

Two independent HeLa cell lines were generated, containing a mutation that truncates the Cwh43 protein at or near Leu533. Western blot analysis indicated that Cwh43 protein was essentially undetectable in these cells (FIGS. 8A-8C). Immunocytochemistry for folate receptor alpha and CD59, two GPI-anchored proteins, showed that loss of Cwh43 expression decreases the association of GPI-anchored proteins with intracellular vesicles (FIGS. 8A-8C). However, flow cytometry indicated that the amount of CD59 on the surface of CWH43 mutant HeLa cell lines was not decreased (FIGS. 8A-8C). Subfractionation of wild-type and CWH43 mutant HeLa cells into aqueous and lipid compartments using Triton X-114, followed by Western blot analysis, revealed that the CWH43 Leu533Ter mutation decreases the association of CD59 with the lipid microdomain fraction where GPI-anchored proteins are typically found (FIG. 1D), even though the amount of CD59 in the total membrane fraction increased slightly. The effect of the CWH43 Leu533Ter mutation on localization of CD59 to the lipid microdomain fraction could be rescued by overexpression of wild-type GFP-Cwh43, but not by GFP-Cwh43-K696fs mutant protein (FIG. 1D). These findings indicate that Cwh43 regulates the membrane targeting of GPI-anchored proteins in human cells. Both of the recurrent iNPH-associated CWH43 deletions disrupt this function.

### Cwh43 Expression in the Mouse Brain

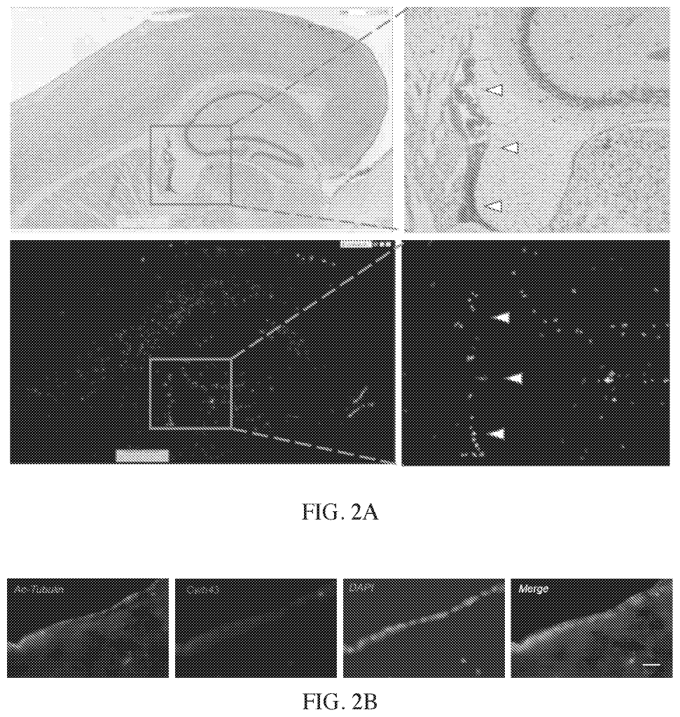

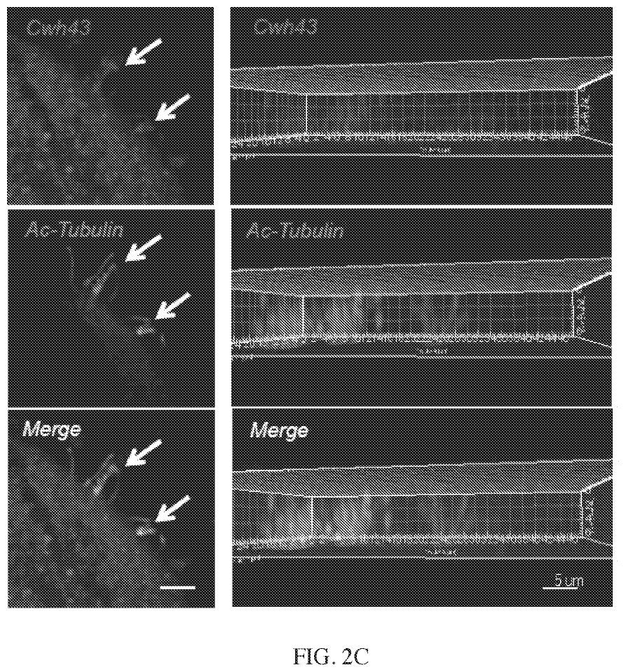

Mouse mRNA in situ hybridization images revealed increased Cwh43 mRNA expression in the choroid plexus, layer CA1-CA3 of the hippocampus, several thalamic nuclei and layer V of the cerebral cortex (FIG. 2A). Using frozen cryostat sections of the mouse brain, it was found that Cwh43 immunoreactivity was concentrated in the ventricular ependymal layer (FIG. 2B) and choroid plexus (FIG. 9). In cultured mouse ependymal cells, Cwh43 immunoreactivity was observed in the soma and in motile cilia (FIG. 2C).

### Effect of Cwh43 Deletion in Mice

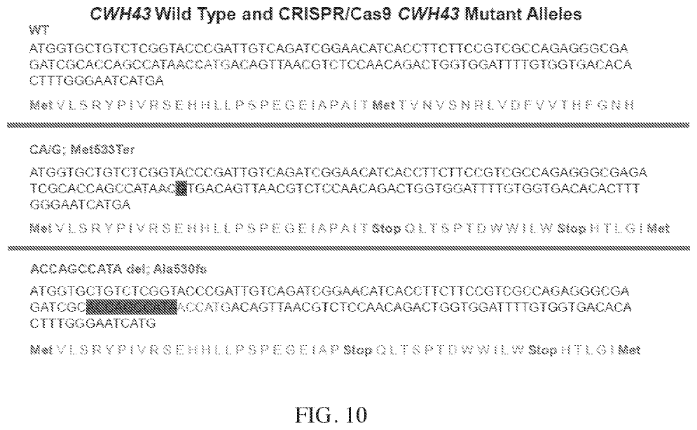

Using CRISPR/Cas9 technology, two independent lines of CWH43 mutant mice were generated (FIG. 10). CWH43^{M533} mice harbor a mutation (Met533Ter) corresponding to human 4:49034669 CA/C; Leu533Ter. A second mouse line (CWH43^{M533/A530}) was generated, harboring one CWH43^{M533} allele and a CWH43 allele that results in termination of Cwh43 at A530, three amino acids before Met533. Heterozygous CWH43^{WT/M533}, homozygous CWH43^{M533/M533}, and compound heterozygous CWH43^{M533/A530} mice appeared grossly normal and were fertile.

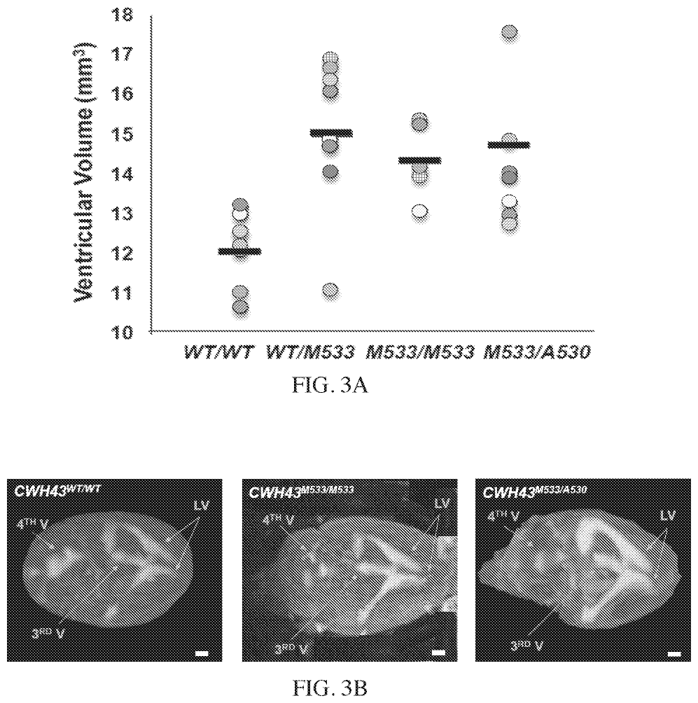

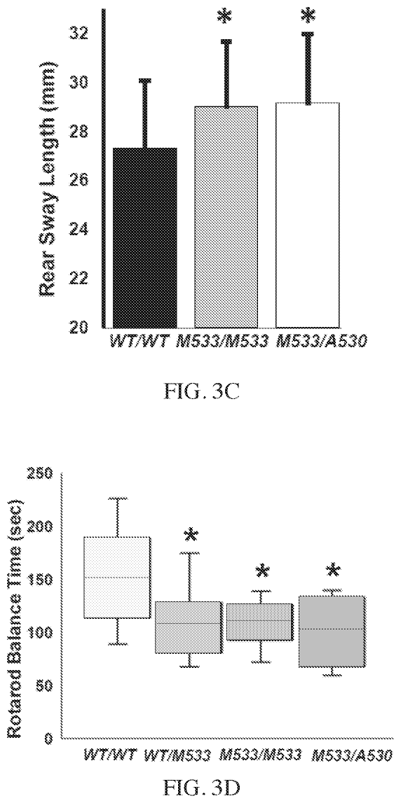

MRI was used to assess ventricular volume in the brains of 6 month old wild-type and CWH43 mutant mice (FIGS. 3A, 3B). When compared to CWH43^{WT/WT} wild-type mice, ventricular volume was increased by approximately 24.2% in CWH43^{WT/M533} heterozygous mice (P<0.0015, n=8, unpaired t-test), 18.3% in CWH43^{M533/M533} homozygous mice (P<0.0014, n=5, unpaired t-test) and 20.8% in CWH43^{M533/A530} compound heterozygous mice (P<0.0064, n=8, unpaired t-test). The brains of CWH43 mutant mice were grossly normal. Injection of fluorescent dextran (70 kDa) into the lateral ventricle resulted in filling of the fourth ventricle within 10 minutes, indicating a patent cerebral aqueduct and communicating hydrocephalus.

Quantitative gait analysis in CWH43 mutant mice at 7 months revealed increased rear leg sway (FIG. 3C, P<0.011, n=9, unpaired t-test) when compared to wild-type mice. Using the rotarod performance test to evaluate balance and coordination, significantly decreased balance times were observed for CWH43W^{T/M533} heterozygous mice (P<0.0405, n=8, unpaired t-test), CWH43^{M533/M533} homozygous mice (P<0.0323, n=7, unpaired t-test) and CWH43^{M533/A530} compound heterozygous mice (P<0.03, n=4, unpaired t-test) when compared to wild-type mice (FIG. 3D).

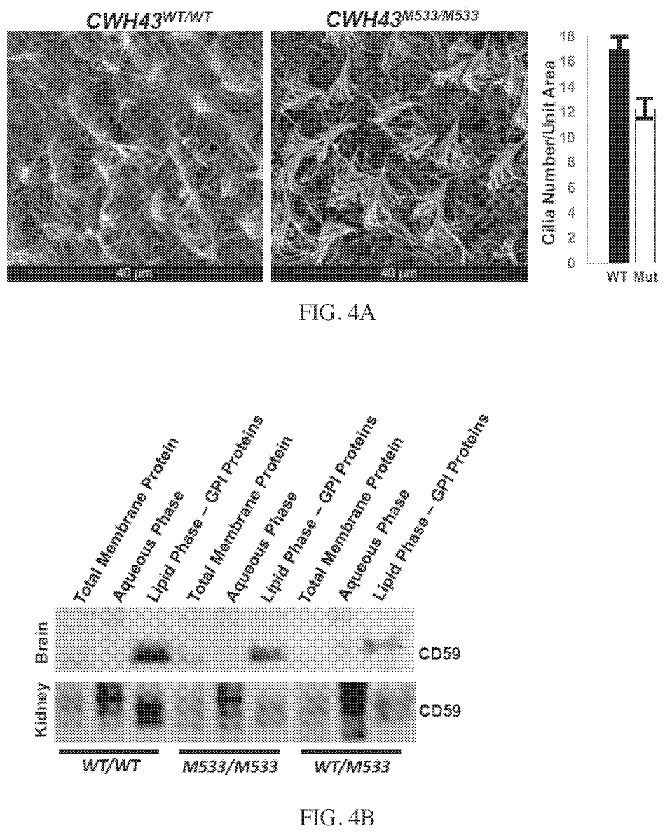

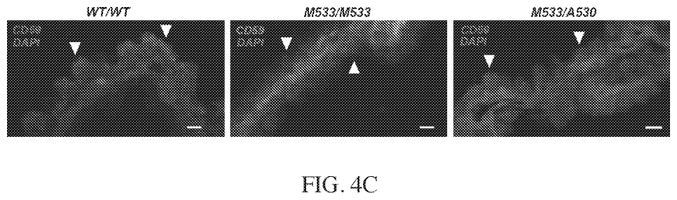

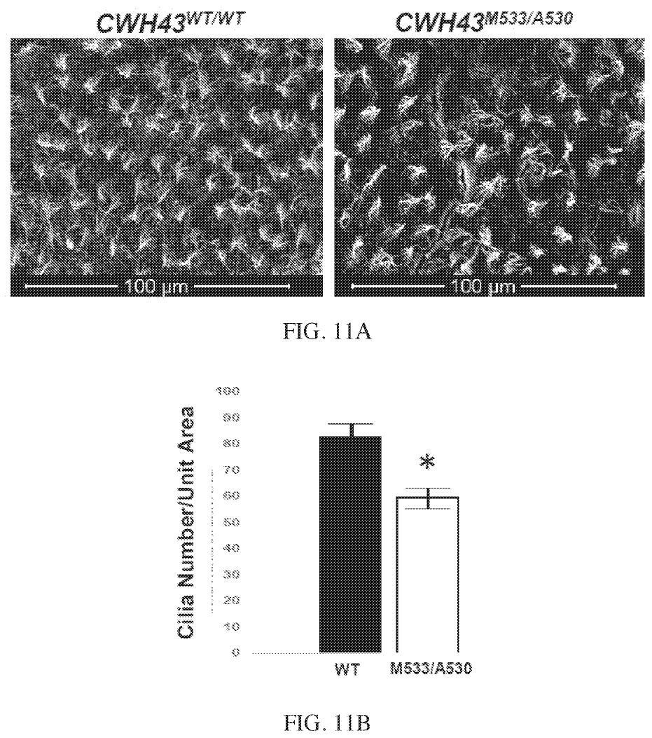

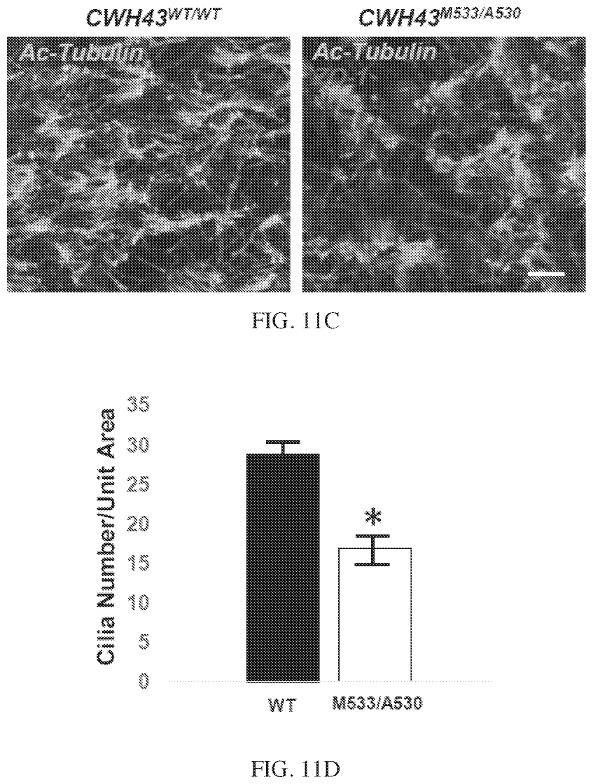

Electron microscopy examination of the mouse brain ventricular surface revealed a 28% decrease in the number of ependymal cilia in CWH43^{M533/M533} homozygous mice (P<0.0037, n=4, unpaired t-test, FIG. 4A) and a 25% decrease in CWH43^{M533/A530} compound heterozygous mice (P<0.0003, n=6, unpaired t-test) when compared to wild-type mice (FIGS. 11A-11D). Immunohistochemistry confirmed the decrease in ependymal cilia in CWH43^{M533/A530} mice (FIGS. 11A-11D).

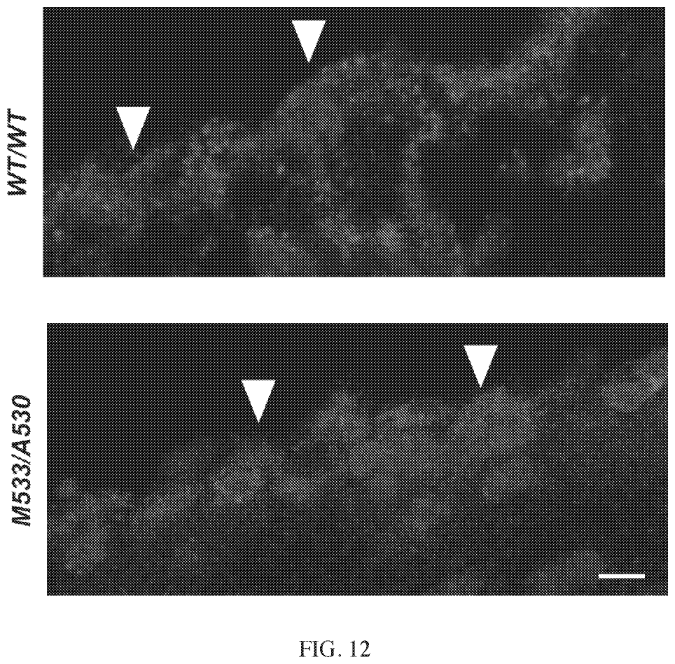

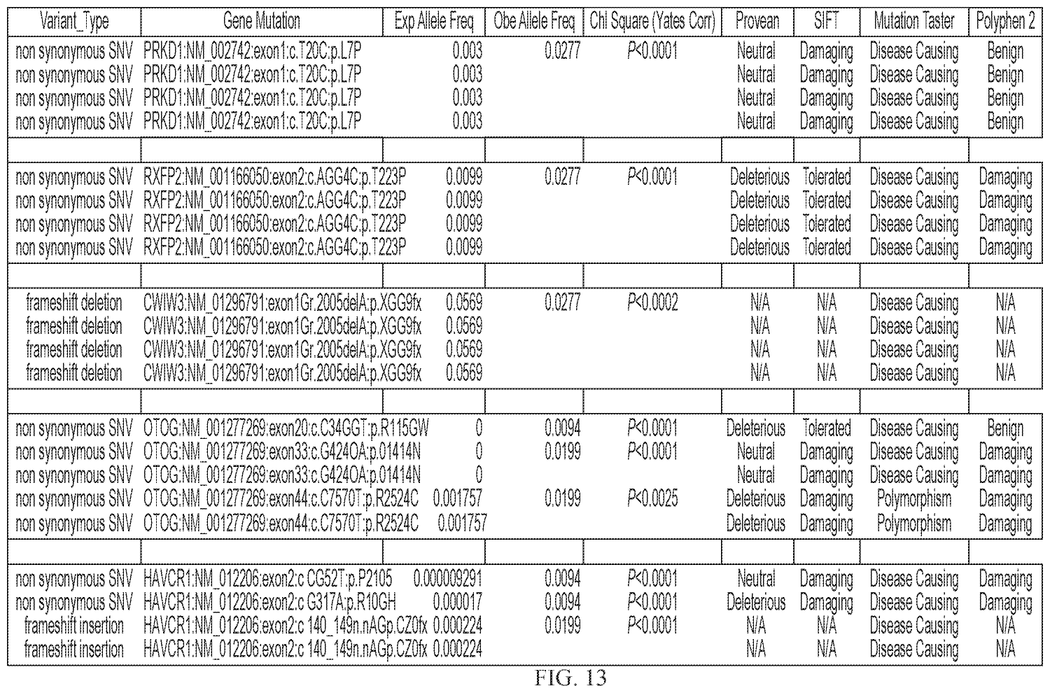

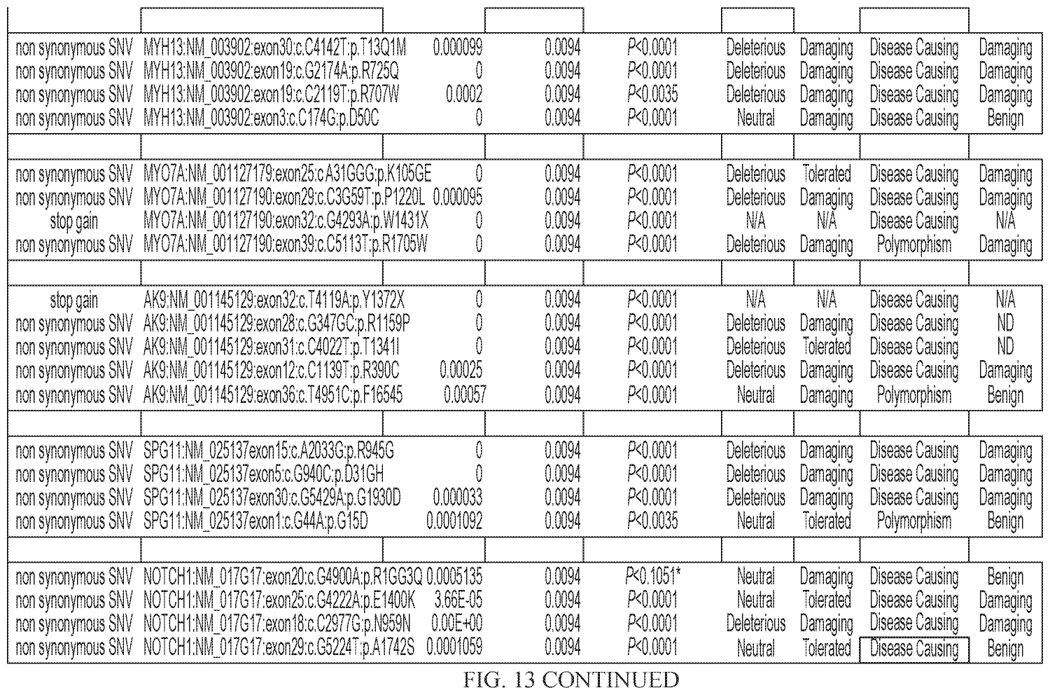

Triton X-114 was used to subfractionate brain and kidney tissues from CWH43^{WT/WT}, CWH43^{WT/M533} and CWH43^{M533/M533} mice into aqueous and lipid compartments. Western blot analysis revealed that CWH43 mutation decreases the association of the GPI-anchored protein, CD59, with the lipid microdomain fraction in heterozygous CWH43^{WT/M533} and homozygous CWH43^{M533/M533} mice in vivo (FIG. 4B). Immunohistochemistry revealed that CWH43 mutation redirected localization of CD59 from the apical membrane to the basal membrane of multi-ciliated choroid plexus (FIG. 4C) and ependymal cells in CWH43^{M533/M533} and CWH43^{M533/A530} mice (FIG. 12).

Most iNPH patients do not report a family history of the disease. However, autosomal dominant transmission of iNPH has been reported. Although a family history of iNPH was not required for entry into this study, 3 of 8 patients with CWH43 deletions reported a family history of iNPH or gait difficulty. Importantly, it was found that humans and mice heterozygous for iNPH-associated CWH43 deletions display an increase in ventricular volume as well as gait and balance dysfunction, consistent with an autosomal dominant pattern of inheritance. The Leu533Ter deletion in CWH43 markedly decreased Cwh43 protein levels, indicating that the increased ventricular size and gait dysfunction observed in humans and in heterozygous CWH43^{WT/M533} mice is due to decreased expression of functional Cwh43 protein rather than a dominant negative effect of mutant Cwh43.

Although iNPH patients appear functionally normal at birth and only develop symptoms after the sixth decade of life, they can develop progressive ventriculomegaly prior to symptom onset. Likewise, CWH43 mutant mice appear normal at birth and show no major deficits through middle age. However, careful testing revealed enlarged ventricles and gait dysfunction during this period. This first animal model of iNPH thus accurately reflects the phenotype and time course of the disease to this point.

Mutations affecting proteins involved in GPI-anchored protein synthesis can cause mental retardation, microcephaly and seizures, while complete disruption of GPI-anchored protein synthesis is lethal. Although Cwh43 modifies the lipid anchor of certain GPI-anchored proteins, it is not required for their basic synthesis. This fact may help to explain the absence of symptoms until late in life in iNPH patients.

Declines in the number of ciliated ventricular cells have been observed in aged asymptomatic individuals with enlarged ventricles and in patients with chronic hydrocephalus. Data described here indicate that decreased Cwh43 function disrupts the trafficking of GPI-anchored proteins and leads to a decrease in the function and/or number of multi-ciliated ventricular cells and ventricular enlargement. In some embodiments, combines with age-related declines in cell number to produce an age-dependent compromise of ventricular multi-ciliated cell function, ventricular enlargement, and iNPH onset at an advanced age.

### Exemplary Sequences

This Table exhibits some exemplary sequences as disclosed by the instant Specification, but is not limiting. This Specification includes a Sequence Listing submitted concurrently herewith as a text file in ASCII format. The Sequence Listing and all of the information contained therein are expressly incorporated herein and constitute part of the instant Specification as filed.

TABLE 2
Exemplary Sequences

| SEQ | | |
|---|---|---|
| ID | | |
| NO: | Sequence* | Description** |
| 1 | See Accompanying Sequence Listing | CWH34 Gene - |
| | | GenBank Accession |
| | | and Version Number: |
| | | NC_000004.12 (NT) |
| 2 | MPSLWREILLESLLGCVSWSLYHDLGPMIYYFPLQTLE | Cwh34 Protein - |
| | LTGLEGFSIAFLSPIFLTITPFWKLVNKKWMLTLLRII | GenBank Accession |
| | TIGSIASFQAPNAKLRLMVLALGVSSSLIVQAVTWWSG | and Version Number: |
| | SHLQRYLRIWGFILGQIVLVVLRIWYTSLNPIWSYQMS | NC_000004.12 (AA) |
| | NKVILTLSAIATLDRIGTDGDCSKPEEKKTGEVATGMA | |
| | SRPNWLLAGAAFGSLVFLTHWVFGEVSLVSRWAVSGHP | |
| | HPGPDPNPFGGAVLLCLASGLMLPSCLWFRGTGLIWWV | |
| | TGTASAAGLLYLHTWAAAVSGCVFAIFTASMWPQTLGH | |
| | LINSGTNPGKTMTIAMIFYLLEIFFCAWCTAFKFVPGG | |
| | VYARERSDVLLGTMMLIIGLNMLFGPKKNLDLLLQTKN | |
| | SSKVLFRKSEKYMKLFLWLLVGVGLLGLGLRHKAYERK | |
| | LGKVAPTKEVSAAIWPFRFGYDNEGWSSLERSAHLLNE | |
| | TGADFITILESDASKPYMGNNDLTMWLGEKLGFYTDFG | |
| | PSTRYHTWGIMALSRYPIVKSEHHLLPSPEGEIAPAIT | |
| | LTVNISGKLVDFVVTHFGNHEDDLDRKLQAIAVSKLLK | |
| | SSSNQVIFLGYITSAPGSRDYLQLTEHGNVKDIDSTDH | |
| | DRWCEYIMYRGLIRLGYARISHAELSDSEIQMAKFRIP | |
| | DDPTNYRDNQKVVIDHREVSEKIHFNPRFGSYKEGHNY | |
| | ENNHHFHMNTPKYFL | |
| 3 | YF | ER Export Signal (AA) |
| 4 | KKKS | ER Retention Signal |
| | | (AA) |
| 5 | NTFYETFKTRSYWLGKSKKKSM | Exemplary Frameshift |
| | | Mutation Insertion |
| | | (AA) |
| 6 | ACCAGCCATA | Exemplary Frameshift |
| | | Mutation Insertion |
| | | (NT) |
| 7 | KKXX | ER retrieval signal |
| | | (AA) |

*Unless otherwise specified, nucleic acid sequences are described 5′ to 3′ and amino acid sequences are described N-terminus to C-terminus
**‘NT’ denotes a nucleic acid sequence; ‘AA’ denotes an amino acid sequence. In the absence of an identifier (e.g., NT, AA), the skilled artisan will readily be able to discern nucleic acid sequences from amino acid sequences by their constituent components. For example, a nucleic acid will only contain those identifiers associated in the art with ribonucleic acid or deoxyribonucleic acid components (e.g., A, C, G, T, U, or other modified base (i.e., nucleotide)) whereas amino acid sequences will contain those identifiers associated in the art with amino acid components (e.g., A, C, D, E, F, G, H, I, K, L, M, N, P, Q, R, S, T, V, W, Y, or other modified amino acid).

### Other Embodiments

Embodiment 1. A method of diagnosing idiopathic normal pressure hydrocephalus (iNPH) in a subject, comprising: (a) measuring in a biological sample from the subject a level of Cwh43 protein; and (b) comparing the level of Cwh43 protein in the sample from the subject with a control level of Cwh43 protein.

Embodiment 2. A method of treating idiopathic normal pressure hydrocephalus (iNPH) in a subject, comprising the diagnostic method of embodiment 1, further comprising: (c) administering to the subject at least one treatment for iNPH when the level of Cwh32 protein is lower than the control level of Cwh43 protein.

Embodiment 3. A method of diagnosing idiopathic normal pressure hydrocephalus (iNPH) in a subject, comprising: (a) analyzing the CWH43 gene in a sample from a subject; and (b) identifying one or more mutations that negatively affect ER export signal of CWH43.

Embodiment 4. A method of treating idiopathic normal pressure hydrocephalus (iNPH) in a subject, comprising the diagnostic method of embodiment 3, further comprising: (c) administering at least one treatment for iNPH if the CWH43 gene product has at least one mutation negatively affecting the ER export signal of the CWH43 gene product.

Embodiment 5. A method of diagnosing idiopathic normal pressure hydrocephalus (iNPH) in a subject, comprising: (a) identifying a truncated Cwh43 protein in a biological sample obtained from a subject.

Embodiment 6. A method of treating idiopathic normal pressure hydrocephalus (iNPH) in a subject, comprising the diagnostic method of embodiment 5, further comprising: (b) administering at least one treatment for iNPH if the isolated Cwh43 is truncated compared to the control Cwh43 protein.

Embodiment 7. The method of embodiment 3, wherein the analysis comprises sequencing the exome of the subject.

Embodiment 8. The method of any one of embodiments 3-4 or embodiment 7, wherein the at least one mutation results in a truncation of the CWH43 gene product within 1, 2, 3, 4, 5, 6, 7, 8, 9, 10 residues, or at residue 533.

Embodiment 9. The method of any one of embodiments 5-6, wherein the isolated Cwh43 protein from the sample from the subject is truncated within 1, 2, 3, 4, 5, 6, 7, 8, 9, 10 residues, or at residue 533.

Embodiment 10. The method of any one of embodiments 2-3 or 7, wherein the at least one mutation in the CWH43 gene which negatively affect the ER export signal is the result of a termination codon being introduced into the gene such that the Cwh43 protein terminates within 1, 2, 3, 4, 5, 6, 7, 8, 9, 10 residues, or at residue 533.

Embodiment 11. The method of any one of embodiments 3, 7-8, or 10, wherein the at least one mutation is listed in Table 1.

Embodiment 12. The method of any one of embodiments 3, 7-8, or 10-11, wherein the CWH43 gene comprises a Lys696AsnfsTer23 mutation.

Embodiment 13. The method of embodiment 1, wherein the measuring comprises an antibody-based assay.

Embodiment 14. The method of embodiment 13, wherein the antibody comprises a detection tag or moiety.

Embodiment 15. The method of any one of embodiments 1-14, wherein the method is done in conjunction with at least one additional method for determining the risk of iNPH in the subject.

Embodiment 16. The method of embodiment 15, wherein the at least one additional method comprises a method selected from: evaluation for symmetric gait disturbances, evaluation for dementia, evaluation for incontinence, and a negative determination of other causes of hydrocephalus.

Embodiment 17. The method of any one of embodiments 2, 4, or 6-13, wherein the at least one treatment comprises, cerebral spinal fluid (CSF) drainage.

Embodiment 18. The method of embodiment 17, wherein the CSF drainage is performed via implantation of a shunt.

Embodiment 19. The method of any one of embodiments 1-18, wherein the at least one treatment comprises, administration of an exogenous Cwh43 protein with at least 70% identity to wild-type Cwh43 protein (SEQ ID NO: 2).

Embodiment 20. The method of embodiment 19, wherein the wild-type Cwh43 protein is human Cwh43 protein.

Embodiment 21. The method of any one of embodiments 1-20, wherein the subject is a mammal.

Embodiment 22. The method of embodiment 21, wherein the mammal is human.

Embodiment 23. The method of any one of embodiments 1-22, wherein the subject is at least 45 years of age.

Embodiment 24. The method of any one of embodiments 1-23, wherein the subject is between about 40 and 55 years of age.

Embodiment 25. The method of any one of embodiments 1-23, wherein the subject exhibits at least one other symptom of iNPH.

Embodiment 26. The method of any one of embodiments 1-23, wherein the subject exhibits at least two other symptoms of iNPH.

Embodiment 27. The method of any one of embodiments 1-23, wherein the subject exhibits at least three other symptoms of iNPH.

In addition to the embodiments expressly described herein, it is to be understood that all of the features disclosed in this disclosure may be combined in any combination (e.g., permutation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, and can make various changes and modifications of the invention to adapt it to various usages and conditions. Thus, other embodiments are also within the embodiments.

### General Techniques

The practice of the subject matter of the disclosure will employ, unless otherwise indicated, conventional techniques of molecular biology (including recombinant techniques), microbiology, cell biology, biochemistry and immunology, which are within the skill of the art. Such techniques are explained fully in the literature, such as, but without limiting, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Equivalents and Scope

It is to be understood that this disclosure is not limited to any or all of the particular embodiments described expressly herein, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents (i.e., any lexicographical definition in the publications and patents cited that is not also expressly repeated in the disclosure should not be treated as such and should not be read as defining any terms appearing in the accompanying claims). If there is a conflict between any of the incorporated references and this disclosure, this disclosure shall control. In addition, any particular embodiment of this disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In the claims articles such as “a,” “an,” and “the” may mean one or more than one unless indicated to the contrary or otherwise evident from the context. Wherever used herein, a pronoun in a gender (e.g., masculine, feminine, neuter, other, etc.) the pronoun shall be construed as gender neutral (e.g., construed to refer to all genders equally) regardless of the implied gender unless the context clearly indicates or requires otherwise. Wherever used herein, words used in the singular include the plural, and words used in the plural includes the singular, unless the context clearly indicates or requires otherwise. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

What is claimed is:

1. A method of diagnosing idiopathic normal pressure hydrocephalus (iNPH) in a subject, comprising:
(a) measuring in a biological sample from the subject a level of Cwh43 protein; and
(b) comparing the level of Cwh43 protein in the sample from the subject with a control level of Cwh43 protein.

2. A method of treating idiopathic normal pressure hydrocephalus (iNPH) in a subject, comprising the diagnostic method of claim 1, further comprising:
(c) administering to the subject at least one treatment for iNPH when the level of Cwh32 protein is lower than the control level of Cwh43 protein.

3. A method of diagnosing idiopathic normal pressure hydrocephalus (iNPH) in a subject, comprising:
(a) analyzing the CWH43 gene in a sample from a subject; and
(b) identifying one or more mutations that negatively affect ER export signal of CWH43.

4. A method of treating idiopathic normal pressure hydrocephalus (iNPH) in a subject, comprising the diagnostic method of claim 3, further comprising:
(c) administering at least one treatment for iNPH if the CWH43 gene product has at least one mutation negatively affecting the ER export signal of the CWH43 gene product.

5. A method of diagnosing idiopathic normal pressure hydrocephalus (iNPH) in a subject, comprising:
(a) identifying a truncated Cwh43 protein in a biological sample obtained from a subject.

6. A method of treating idiopathic normal pressure hydrocephalus (iNPH) in a subject, comprising the diagnostic method of claim 5, further comprising:
(b) administering at least one treatment for iNPH if the isolated Cwh43 is truncated compared to the control Cwh43 protein.

7. The method of claim 3, wherein the analysis comprises sequencing the exome of the subject.

8. The method of any one of claims 3-4 or claim 7, wherein the at least one mutation results in a truncation of the CWH43 gene product within 1, 2, 3, 4, 5, 6, 7, 8, 9, 10 residues, or at residue 533.

9. The method of any one of claims 5-6, wherein the isolated Cwh43 protein from the sample from the subject is truncated within 1, 2, 3, 4, 5, 6, 7, 8, 9, 10 residues, or at residue 533.

10. The method of any one of claim 2-3 or 7, wherein the at least one mutation in the CWH43 gene which negatively affect the ER export signal is the result of a termination codon being introduced into the gene such that the Cwh43 protein terminates within 1, 2, 3, 4, 5, 6, 7, 8, 9, 10 residues, or at residue 533.

11. The method of any one of claim 3, 7-8, or 10, wherein the at least one mutation is listed in Table 1.

12. The method of any one of claim 3, 7-8, or 10-11, wherein the CWH43 gene comprises a Lys696AsnfsTer23 mutation.

13. The method of claim 1, wherein the measuring comprises an antibody-based assay.

14. The method of claim 13, wherein the antibody comprises a detection tag or moiety.

15. The method of any one of claims 1-14, wherein the method is done in conjunction with at least one additional method for determining the risk of iNPH in the subject.

16. The method of claim 15, wherein the at least one additional method comprises a method selected from: evaluation for symmetric gait disturbances, evaluation for dementia, evaluation for incontinence, and a negative determination of other causes of hydrocephalus.

17. The method of any one of claim 2, 4, or 6-13, wherein the at least one treatment comprises, cerebral spinal fluid (CSF) drainage.

18. The method of claim 17, wherein the CSF drainage is performed via implantation of a shunt.

19. The method of any one of claims 1-18, wherein the at least one treatment comprises, administration of an exogenous Cwh43 protein with at least 70% identity to wild-type Cwh43 protein (SEQ ID NO: 2).

20. The method of claim 19, wherein the wild-type Cwh43 protein is human Cwh43 protein.

21. The method of any one of claims 1-20, wherein the subject is a mammal.

22. The method of claim 21, wherein the mammal is human.

23. The method of any one of claims 1-22, wherein the subject is at least 45 years of age.

24. The method of any one of claims 1-23, wherein the subject is between about 40 and 55 years of age.

25. The method of any one of claims 1-23, wherein the subject exhibits at least one other symptom of iNPH.

26. The method of any one of claims 1-23, wherein the subject exhibits at least two other symptoms of iNPH.

27. The method of any one of claims 1-23, wherein the subject exhibits at least three other symptoms of iNPH.
